(19) United States
(12) Patent Application Publication
(10) Pub. No.: US 2023/0374011 A1
(43) Pub. Date: Nov. 23, 2023

(54) SUBSTITUTED TRICYCLIC COMPOUNDS

(71) Applicant: Unichem Laboratories Ltd., Maharashtra (IN)

(72) Inventors: Dhananjay G. Sathe, Maharashtra (IN); Dnyaneshwar V. Gawas, Goa (IN); Gorakhnath S. Yellol, Maharashtra (IN)

(21) Appl. No.: 18/030,583

(22) PCT Filed: Oct. 6, 2021
(86) PCT No.: PCT/IB2021/059150

§ 371 Date: Apr. 6, 2023

(30) Foreign Application Priority Data
Oct. 8, 2020 (IN) 202021043852

(51) Int. Cl. C07D 471/14

(52) U.S. Cl. CPC C07D 471/14

(57) ABSTRACT

The invention provides a compound of formula (I) or a pharmaceutically acceptable salt thereof; wherein Q is a group of formula Q1 or Q2; wavy bond represents the points of attachment; wherein R₁ is —NR^{a}R^{b}; R₂ is hydrogen or a C₁-C₁₀ alkyl group; R^{a} and R^{b} independently represent hydrogen or a C₁-C₁₀ alkyl group, and use of these compounds as kinase inhibitors and compositions comprising the compounds of the present invention.

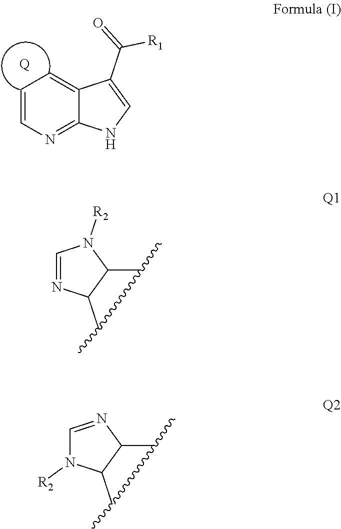

[symbol]

(wavy bond)

### FIELD OF THE INVENTION

The present invention relates to a novel compound of Formula (I) or a pharmaceutically acceptable salt thereof. The invention also relates to a process of preparation of compounds of present invention, use of these compounds as kinase inhibitors and compositions comprising the compounds of present invention.

### BACKGROUND OF INVENTION

Protein kinases (PTKs) are enzymes that regulate the biological activity of proteins by phosphorylation of specific amino acids with ATP as the source of phosphate, thereby inducing a conformational change from an inactive to an active form of the protein. They serve to orchestrate the activity of almost all cellular processes and therefore are key regulators of cell function. Kinases are particularly prominent in signal transduction and co-ordination of complex functions such as the cell cycle. Sometimes protein kinasesare classified based on the substrates that they phosphorylate. For example,serine/threonine protein kinases phosphorylate serine or threonine amino acid residues whereas tyrosine kinase phosphorylate tyrosine amino acid residues.

Tyrosine kinases are important mediators of the signal transduction process, leading to cell proliferation, differentiation, migration, metabolism and programmed cell death. They are implicated in several steps of neoplastic development and progression. Tyrosine kinase signalling pathways normally prevent deregulated proliferation or contribute to sensitivity towards apoptotic stimuli. Janus kinases (referred to as JAK) are tyrosine kinases that are involved in transduction of cytokine signalling from membrane receptors to signal transducer and activator of transcription (STAT) factors. Cytokines play key roles in controlling cell growth and the immune response. Many cytokines function by binding to and activating type I and type II cytokine receptors. These receptors in turn rely on the Janus kinase (JAK) family of enzymes for signal transduction. Currently, there are four known mammalian JAK family members: JAK1 (Janus kinase-1), JAK2 (Janus kinase-2), JAK3 (also known as Janus kinase leukocyte; JAKL; L-JAK and Janus kinase-3) and TYK-2 (also known as protein-tyrosine kinase 2). Mutation or abnormal functioning of JAK may lead to signalling pathways that are genetically or epigenetically altered imparting selection advantage to cancer cells. Such abnormalities may also cause diseases resulting from inappropriate activation of the immune and nervous systems such as inflammatory conditions, autoimmune diseases, proliferative diseases, transplantation rejection, diseases involving impairment of cartilage turnover, congenital cartilage malformations, and/or diseases associated with hypersecretion of IL6.

Kinase mediated diseases are prevented or treated by inhibiting their activities. JAK inhibitors interfere with the JAK-STAT signalling pathway. Hence drugs that inhibit the activity of these Janus kinases block cytokine signalling that are effective against immune response (Current Opinion in Pharmacology. 12 (4): 464-70).

U.S. Pat. No. RE41783 discloses some pyrrolopyrimidine compounds that are JAK inhibitors, more specifically JAK3 inhibitors. Tricyclic and triazolopyridine compounds disclosed in U.S. Pat. Nos. 8,962,629 and 8,088,764 respectively are specific JAK1 inhibitors whereas azetidinederivatives disclosed in U.S. Pat. No. 8,158,616 are mixed JAK1 and JAK2 inhibitors. While these JAK inhibitors have been shown to be satisfactory, more effective and potent treatment is required for JAK related diseases. There remains a need to study and identify new compounds that may be effective in treating diseases due to mutations and malfunctioning of Janus Kinase.

Therefore, the object of the present invention is to provide an alternative in the form of novel compounds effective in prevention and/or treatment of diseases arising due to abnormal functioning of JAK. It is further an object to provide a process to prepare these novel compounds, their pharmaceutical formulations and a method of treatment of diseases arising due to JAK abnormalities using compounds of the present invention. Yet another object of the invention is to provide cheap and affordable JAK inhibitors. Another object of the invention is to provide a method to cure or reduce the effect of diseases arising due to abnormal functioning of JAK.

### SUMMARY OF THE INVENTION

The present invention relates to a compound of formula (I)

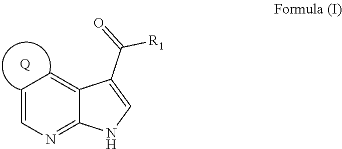

or a pharmaceutically acceptable salt thereof;

wherein Q is a group of formula Q1 or Q2;

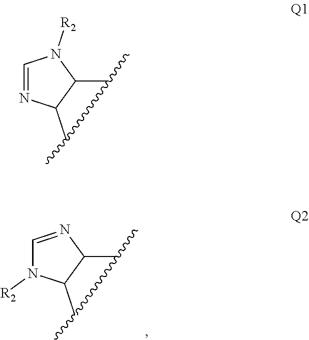

[symbol] (wavy bond) represents the points of attachment;

wherein R₁ is —NR^{a}R^{b};

R₂ is hydrogen or a C₁-C₁₀ alkyl group;

R^{a} and R^{b} independently represent hydrogen or a C₁-C₁₀ alkyl group.

Another embodiment of the present invention provides a specific preferred compound of formula (I) selected from the group consisting of:
N,1-dimethyl-1,6-dihydroimidazo[4,5-d]pyrrolo[2,3-b]pyridine-8-carboxamide;
N-ethyl-1-methyl-1,6-dihydroimidazo[4,5-d]pyrrolo[2,3-b]pyridine-8-carboxamide;
1-methyl-N-propyl-1,6-dihydroimidazo[4,5-d]pyrrolo[2,3-b]pyridine-8-carboxamide;
1-methyl-N-(propan-2-yl)-1,6-dihydroimidazo[4,5-d]pyrrolo[2,3-b]pyridine-8-carboxamide;
N-methyl-1,6-dihydroimidazo[4,5-d]pyrrolo[2,3-b]pyridine-8-carboxamide;
N-ethyl-1,6-dihydroimidazo[4,5-d]pyrrolo[2,3-b]pyridine-8-carboxamide;
N-(propan-2-yl)-1,6-dihydroimidazo[4,5-d]pyrrolo[2,3-b]pyridine-8-carboxamide;
N,3-dimethyl-3,6-dihydroimidazo[4,5-d]pyrrolo[2,3-b]pyridine-8-carboxamide;
N-ethyl-3-methyl-3,6-dihydroimidazo[4,5-d]pyrrolo[2,3-b]pyridine-8-carboxamide;
3-methyl-N-propyl-3,6-dihydroimidazo[4,5-d]pyrrolo[2,3-b]pyridine-8-carboxamide;
3-methyl-N-(propan-2-yl)-3,6-dihydroimidazo[4,5-d]pyrrolo[2,3-b]pyridine-8-carboxamide;
N-methyl-3,6-dihydroimidazo[4,5-d]pyrrolo[2,3-b]pyridine-8-carboxamide;
N-ethyl-3,6-dihydroimidazo[4,5-d]pyrrolo[2,3-b]pyridine-8-carboxamide; and
N-(propan-2-yl)-3,6-dihydroimidazo[4,5-d]pyrrolo[2,3-b]pyridine-8-carboxamide; or a pharmaceutically acceptable salt thereof.

Another embodiment of the present invention provides a specific preferred compound of formula (I) which is N-(propan-2-yl)-1,6-dihydroimidazo[4,5-d]pyrrolo[2,3-b]pyridine-8-carboxamide hydrochloride.

Another embodiment of the present invention provides a specific preferred compound of formula (I) which is N-(propan-2-yl)-3,6-dihydroimidazo[4,5-d]pyrrolo[2,3-b]pyridine-8-carboxamide hydrochloride.

Another embodiment of the present invention provides a process to prepare a compound of formula I or its pharmaceutical acceptable salts.

In another embodiment of the invention there is provided a pharmaceutical composition comprising a compound of formula (I) or a pharmaceutically acceptable salt thereof.

In a further embodiment of the invention there is provided a compound of formula (I) or a pharmaceutically acceptable salt thereof or a composition comprising such a compound or salt thereof for use in the treatment or prevention of a disease or condition that is caused by an abnormal functioning of a kinase, especially a Janus kinase.

In another embodiment, the invention provides use of a compound of formula (I) or a pharmaceutically acceptable salt thereof or a composition comprising such a compound or salt thereof for the manufacture of a medicament for use in the treatment or prevention of a disease or condition that is caused by an abnormal functioning of a kinase, especially a Janus kinase.

In a further embodiment, the invention provides a method of treating or preventing a disease or condition that is caused by an abnormal functioning of a kinase, especially a Janus kinase, in a subject need thereof, the method comprising administering to the subject a therapeutically effective amount of a compound of formula (I) or a pharmaceutically acceptable salt thereof or a composition comprising such a compound or salt thereof.

### DETAILED DESCRIPTION OF THE INVENTION

The following reaction schemes illustrates the preparation of the compounds of the present invention.

Scheme I illustrates the preparation of a compound of formula (I) wherein Q, R₁ and R₂ are defined as above and L₁ and L₂ represent X or leaving groups. X may be a leaving group which is either the same as that of L₁ or L₂ or other than that of L₁ and L₂. X may also be a group that can be easily substituted by or converted to —COR¹.

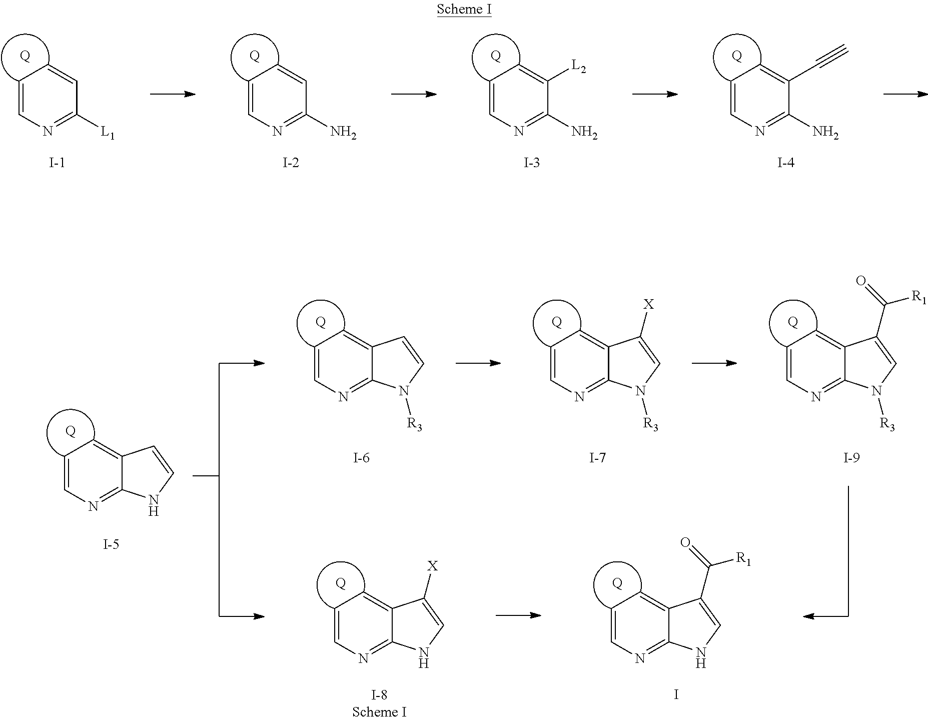

According to an aspect of the present invention the leaving group L₁, L₂ or X is one which can be easily replaced by the desired group or atom. The leaving group may be selected from halogen atoms, alkoxy and sulfonyloxy groups. Examples ofsulfonyloxy groups include, but are not limited to, alkylsulfonyloxy groups (for example methyl sulfonyloxy (mesylate group) and trifluoromethylsulfonyloxy (triflate group)) and arylsulfonyloxy groups (for example/toluenesulfonyloxy (tosylate group) and/-nitrosulfonyloxy (nosylate group)). For the purpose of the present invention L₂ and X may be particularly selected from halogens such as bromo, chloro or iodoand a triflate group. The selection of X will be well within the understanding and knowledge of the skilled person.

In the above reactions of Scheme La compound of formula I-1 is converted into a compound of formula I-2 by a displacement reaction of a compound of formula I-1 with ammonia solution ina suitable solvent,such as water, THF, 1,4-Dioxane, Dimethyl formamide (DMF), Dimethyl sulfoxide (DMSO) orAcetonitrile (ACN),or mixture(s) thereforeat a temperature ranging from 45° C. to 120° C. for 0.5 hr to 20 hrs to form a compound of formula I-2.

A compound of formula I-2 is converted to a compound of formula I-3 by reacting a compound of formula I-2 with a triflating agent such as trifluoromethanesulfonic anhydride or a halogenating agent in a suitable solvent such as acetonitrile, chloroform or tetrahydrofuran at a temperature ranging from −20° C. to the refluxing temperature for a time period between about 1 hour to about 10 hours.

A halogenating agent according to the present invention is a reagent that is a source of halogen. For example the agent may be a chlorinating agent such as chlorine, thionyl chloride, N-Chlorosuccinimide, Oxalyl Chloride or a brominating agent such as bromine, N-Bromosuccinimide, Carbon Tetrabromide or an iodinating agent such as Iodine, Hydriodic Acid or N-Iodosuccinimide. The halogenating agent may be selected according to the knowledge and understanding of skilled person.

A Sonogashira reaction with a compound of formula I-3 and an acetylene derivativeusing a suitable catalyst provides a compound of formula I-4. The reaction conditions for a Sonogashira reaction vary depending on the starting material, the solvent and the transition metal catalyst. The reaction conditions are not limited in particular as long as they are conditions similar to the present reactions, and the methods well known to those skilled in the art can be used. Examples of preferred solvents include acetonitrile, tetrahydrofuran, 1,4-dioxane, 1,2-dimethoxyethane, benzene, toluene, xylene, 1-methyl-2-pyrrolidone, N,N-dimethylformamideand dimethylsulfoxide, dichloromethane or mixture thereof. The reaction temperature should be a temperature that is sufficient to complete the coupling reaction, and is preferably from room temperature to 100° C. The present reaction can be carried out under an inert gas atmosphere, and also under a nitrogen or an argon gas atmosphere. Under the preferred reaction conditions, this reaction is completed in 1 to 24 hours. The transition metal catalyst is preferably a palladium complex. Examples of palladium complexes include, but not limited to palladium(II) acetate, dichlorobis(triphenylphosphine)palladiu-m(II), tris(dibenzylideneacetone)dipalla- -dium(0) and tetrakis(triphenylphosphine)palladium(0). Furthermore, in the present reaction, a phoshorous chelating agent such as triphenylphosphine, tri-o-tolylphosphine or tri-tert-butylphosphine may be added in order to obtain satisfactory results. Further the reaction may be accelerated using a metal halide or a quaternary ammonium salt or other such salts, preferably copper(I) iodide, lithium chloride, tetrabutylammoniumfluoride or silver(I) oxide. Preferred results can also be obtained in the presence of a base; the base used is not limited in particular as long as it is used in a coupling reaction similar to the present reaction Examples of such bases include, but not limited to diethylamine, triethylamine, N,N-diisopropylethylamine, piperidineand pyridine.

A compound of formula I-4 can readily undergo 5-endo-dig cyclization in the presence of a base or transition metal catalyst in the presence of a suitable solvent such as alcoholic solvents or THF or DMA to provide a compound of formula I-5. Exemplarily the base may be selected from Potassium tert-butoxide, Lithium hydride, Lithium Aluminium hydride and n-butyl lithium and the transition metal catalyst may be selected from Palladium and a copper catalyst.

A compound of formula I-5 can be optionally protected by treating it with a protecting group to provide a compound of formula I-6.

Exemplarily a compound of formula I-5 is converted to the corresponding compound of formula I-6, wherein R₃ is benzenesulfonyl or benzyl, by treating the compound of formula I-5 with benzenesulfonyl chloride, benzylchloride or benzylbromide in the presence of a base, such as sodium hydride or potassium carbonate, and a polar aprotic solvent, such as dimethylformamide or tetrahydrofuran. The reaction mixture is stirred at a temperature between about 0° C. to about 70° C., preferably about 30° C., for a time period between about 1 hour to about 3 hours, preferably about 2 hours.

R₃ is a protecting group such asbenzenesulfonyl, substituted benzenesulfonyl,methylsulfonyl, benzyl or carbamate protecting groups such as Hoc (t-Butyloxycarbonyi) and CBz (carboxybenzyl) or other groups such as benzoyl, iso-butanoyl, acetyl, phenoxya.cetyl, 4-(t-butyl)benzoyl, 4-(t-butyl)phenoxyacetyl, 4-(methoxy)benzoyl, 2-(4-nitro-phenypethyloxycarbonyl, 2-(2,4-dinitrophenyl)ethyloxy-carbonyl, 9-fluorenylmethoxycarbonyl, diphenylcarbamoyl or formamidine groups. Particularly preferred are the benzoyl, isobutanoyl, 4-(t-butyl)benzoyl, 2-4-nitro-i5 phenyl)ethyloxycarbonyl, 2-(2,4-dinitrophenypethyl-oxycarbonyl, 9-fluorenylmethoxycarbonyl, 4-(methoxy)-benzoyl or para-(t-butyl)phenoxyacetyl, para-nitrophenyl-2-ethyloxycarbonyi group or 2-N-acetyl with the 6-0-diphenylcarbamoyl group.

Compounds of formula I-5 and I-6 can be converted to a compound of formula I-8 and I-7, respectively in a similar way as the process described for the preparation of a compound of formula I-3.

Compounds of formula I-8can be converted into compounds of formula (I) by a process known to the person skilled in the art. Such process may include converting X of formula I-8 directly to an amide group or via formation of ester, anhydride, aldehyde, ketone, cyanide, acid or any such group which can be converted to an amide group which is well within the understanding and knowledge of the skilled person.

For example when X is converted to an ester group and successively converted to an amide,compounds of formula I-8can betreated withan esterifying agent in the presence ofa base in a polar aprotic solvent like THF, 1,4-Dioxane, DMF, DMSO and ACN at −75° C. to 100° C. temperature for 0.5 hr to 20 hrs which leads to formation of ester derivative. The ester derivative on reaction with a trialkylaluminium (like, trimethylaluminium) and required amine derivatives or ammonia solution in the presence of solvents like Toluene, chloroform, methanol, ethanol, THF, 1,4-Dioxane, DMF, DMSO and ACN at −10° C. to 100° C. temperature for 0.5 hr to 20 hrs gives an amide having formula I.

A compound of formula I-7 can be converted to a compound of formula I-9using a similar process that may be used for conversion of a compound of formula I-8 to a compound of formula I.

A compound of formula I-9 can be converted into a compound of formula I by cleaving the protecting group R₃. Protecting groups of a compound of formula I-9 can be cleaved by deprotecting agents as understood by the skilled person to obtain a compound of formula I. Examples of deprotecting agents for an amino protective group are acids such as trifluoroacetic acid, trichloroacetic acid, dichloroacetic acid p-toluenesulfonic acid or bases such as alkali or alkaline bases. For example, fora compound of formula I-9 wherein R₃ is benzenesuifonyl, the deprotection is carried out by treating I-9 with an alkali base, such as sodium hydroxide or potassium hydroxide, sodium carbonate, potassium carbonate, potassium tert-butoxide, sodiumtert-butoxide in an alcohol solvent, such as methanol or ethanol, or mixed solvents, such as alcohol/tetrahydrofuran or alcohol/water. The reaction is carried out at room temperature or to reflux temperature for a time period between about 15 minutes to about 1 hour, preferably 30 minutes. When R₃ is benzyl, deprotection is either conducted by treating I-9 with sodium in ammonia at a temperature of about −78° C. for a time period between about 15 minutes to about 1 hour or by using hydrogen and a catalyst, such as palladium hydroxide on carbon, Pd/C, Raney Nickel, Raney Nickel in combination with NH₂—NH₂ or Hydrogen. Other suitable deprotecting agents are Lewis acids, such as, for example boron trifluorideetherateor zinc bromide in dichloromethane/isopropanol, aq. HCl, aq. HBr, HBr in acetic acid, sulfuric acid.

Scheme II also illustrates the preparation of a compound of formula (I) wherein Q, R₁R₂, R₃ and X are defined as above. R is represent alkoxy (—OR) or CX₃, Z is NO₂.

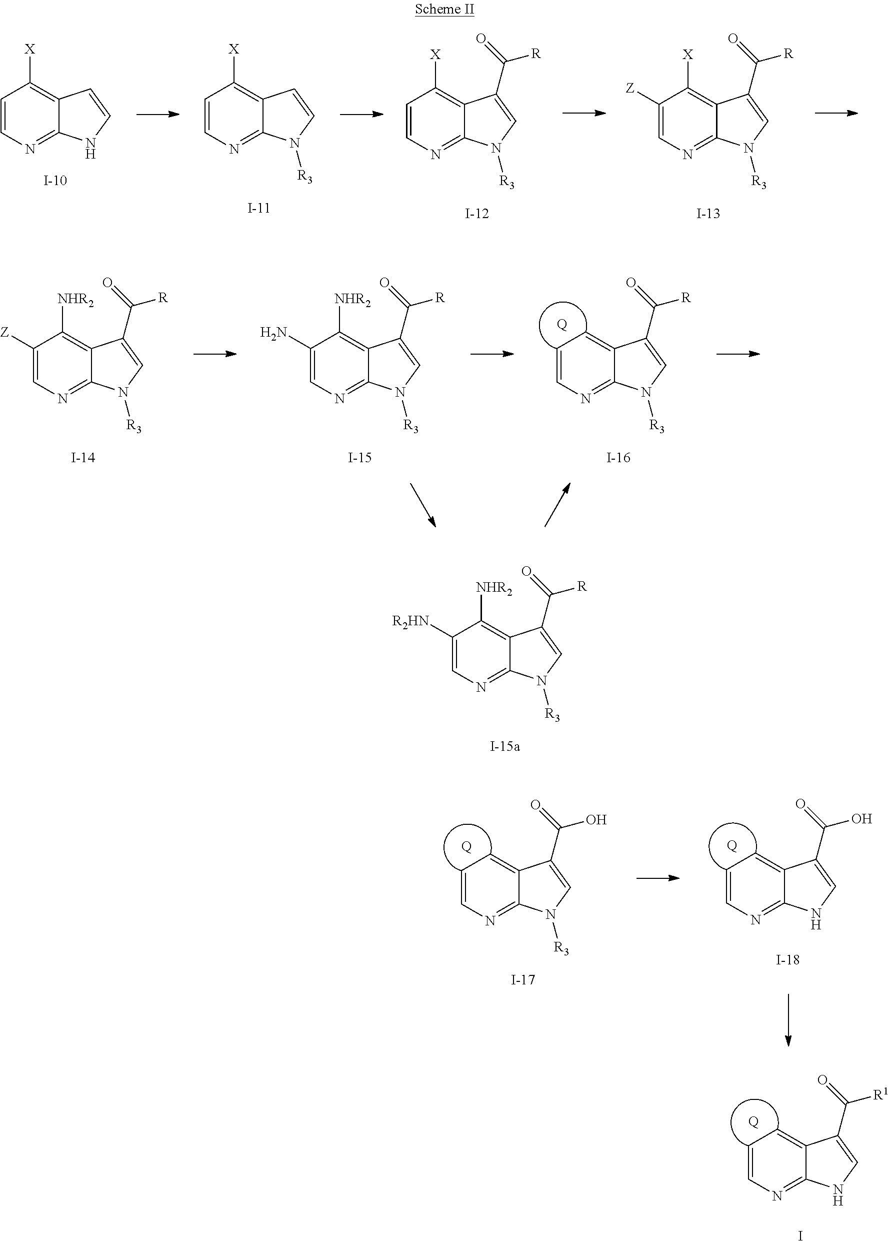

In the above reactions of Scheme II, a compound of formula I-10can be converted to the corresponding compound of formula I-11, by treating the compound of formula I-10 with protecting group R₃ such as benzenesulfonyl chloride, benzylchloride or benzylbromide in the presence of a base, such as sodium hydride, potassium carbonate, sodium hydroxide, potassium hydroxide or cesium carbonate or alkyl lithium such as n-butyl lithium, secondary butyl lithium, tertiary butyl lithium or lithium diisopropyl amide. Such reaction may be carried out in solvent such as dimethylformamide, dimethylacetamide, tetrahydrofuran,hexamethyl phosphoramide,dimethyl sulfoxide,1,4-Dioxane,acetonitrile, water, dichloromethane, Toluene, DMSO or mixture(s) therefore. The reaction mixture is stirred at a temperature between about 0° C. to about 70° C., preferably about 10° C., for a time period between about 1 hour to about 10 hours, preferably about 4 hours.

R₃ is a protecting group defined as above.

Compounds of formula I-11 can be converted to a compound of formula I-12 by reacting a compound of formula I-11 with an acylating agent such as trifluoroacetic anhydride, trichloroacetyl chloride, acid halides, acid anhydrides in a suitable solvent such as acetonitrile, chloroform,n-methyl pyrrolidone, toluene, tetrahydrofuran, dimethylformamide, dimethylsulfoxide, dimethylacetamide 1,4-Dioxane chlorinated alkyl or aryl solvents such as dichloromethane or chlorobenzene, dichlorobenzene or dichloroethane or mixture(s) therefore at a temperature ranging from −20° C. to the refluxing temperature for a time period between about 1 hour to about 15 hours preferably at 65-75° C. for 4-5 hours.

A compound of formula I-12can be converted to a compound of formula I-13by treating compound of formula I-12 with nitrating agents such as alkyl ammonium nitrate for example, tetrabutyl ammonium nitrate or tetramethyl ammonium nitrate and using trifluoroacetic anhydride in solvents such as dichloromethane, toluene, acetonitrile, tetrahydrofuran, chlorobenzene, nitrobenzene, dichloroethane 1,4-Dioxane, acetonitrile, water, dimethylsulfoxide or mixture(s) therefore, at a temperature ranging from −10° C. to 100° C. for a time period between about 1 hour to about 30 hours preferably for 5 hours.

A compound of formula I-13can be converted to a compound of formula I-14by reaction with ammonia or with primary amines such as methyl amine, ethyl amine, isopropyl amine,n-propyl amine, isobutylamine or n-butylamine in suitable solvents such as tetrahydrofuran, dichloromethane, 1,4 dioxane, toluene, dimethylformamide, water, alcoholic solvents, DMSO, acetonitrile or mixture(s) thereof at a temperature ranging from −10° C. to the refluxing temperature for a time period between about 1 hour to about 25 hours, preferably for 8-10 hours.

A compound of formula I-14can be converted to compound of formula I-15 by reduction of nitro group using metal catalyst such as palladium on carbon, Raney nickel, Raney nickel in combination with NH₂—NH₂ or Hydrogen, iron/ammonium chloride, platinum on carbon, zinc/ammonium chloride, Fe/AcOH or sodium dithionite in suitable alcoholic solvents such as methanol, ethanol or water or cyclic/acyclic ethers such as tetrahydrofuran or 1,4-dioxane or acetonitrile and water or in mixture of suitable alcoholic solvents such as methanol, ethanol, or cyclic/acyclic ethers such as tetrahydrofuran or 1,4-dioxane or acetonitrile and water at temperature ranging from −10° C. to reflux temperature, preferably at room temperature for time period of 1 to 10 hours.

A compound of formula I-15 is optionally converted to compound of formula I-15a by treating compound of formula I-15 with alkylating agents or treating with aldehydes, ketones followed by reduction by themethodsknown to person skilled in the art.

A compound of formula I-15 or I-15a can be converted to compound of formula I-16 by cyclization methodsusing reagents such as triethylorthoformate and acid catalyst viz para toluene sulphonic acid or dimethylformamide or formic acid and metal catalyst such as zinc acetate, using solvents such as toluene, halobenzene such as chlorobenzene, 1,2 dichlorobenzene, dimethylformamide, dimethylacetamide, tetrahydrofuran, acetonitrile, 1,4-dioxane, water, acetic acid, formic acid, formamide or mixture(s) thereofat a temperature ranging from room temperature to reflux temperature preferably at 0° C.-100° C. for period of 1 to 10 hours preferably for 5 hours.

A compound of formula I-16can be converted to compound of formula I-17 by hydrolysis using alkali hydroxide such as sodium hydroxide, potassium hydroxide or lithium hydroxide or aqueous solution thereof or any other reagents as understood by the skilled person in suitable alcoholic solvents such as methanol or ethanol or water or in mixture of suitable alcoholic solvents such as methanol, ethanol, propanol, butanol, iso-butanol or cyclic/acyclic ethers such as tetrahydrofuran or 1,4-Dioxane or acetonitrile and water to obtain a compound of formula I-17, ata temperature ranging from room temperature to reflux temperature preferably at a temperature 80° C. for time period of 30 minutes to 10 hours.

A compound of formula 1-17 can be converted into a compound of formula I-18 by cleaving the protecting group R₃. Protecting groups of a compound of formula I-17 can be cleaved by deprotecting agents as understood by the skilled person to obtain a compound of formula I. Examples of deprotecting agents for an amino protective group are acids such as trifluoroacetic acid, trichloroacetic acid, dichloroacetic acid p-toluenesulfonic acid, HCl, HBr, H₂SO₄ or bases such as alkali or alkaline bases. For example, for a compound of formula I-17 wherein R₃ is benzenesulfonyl, the deprotection is carried out by treating I-17 with an alkali base, such as sodium hydroxide or potassium hydroxide, sodium carbonate, potassium carbonate, cesium carbonate in an alcohol solvent, such as methanol or ethanol, or mixed solvents, such as alcohol/tetrahydrofuran or alcohol/water, MDC, THF, toluene, CAN, water or mixture(s) thereof. The reaction is carried out at room temperature to reflux temperature for a time period between about 15 minutes to about 1 hour, preferably 30 minutes. When R₃ is benzyl, deprotection is either conducted by treating I-17 with sodium in ammonia at a temperature of about −78° C. for a time period between about 15 minutes to about 10 hour or by using hydrogen and a catalyst, such as palladium hydroxide on carbon, Pd/C in ether solvents such as tetrahydrofuran and alcohol such as tert-butanol, MDC, THF, toluene, CAN, water or mixture(s) thereof. Other suitable deprotecting agents are Lewis acids, such as, for example boron trifluorideetherate or zinc bromide in dichloromethane/isopropanol. HCl, HBr, H₂SO₄.

A compound of formula I-18 can be converted to compound of formula I by reaction of acid derivative (Formula 1-18) with chlorinating agent such as thionyl chloride, oxalylchlorideusing mixture of solvents such as ditneth.ylformamide, dimethyl acetamide, dichloromethane, dichloroethane, tetrahydrofuran, benzene, toluene, halobenzene viz. 1,2 dichlorobenzene or acetonitrile , at a temperature ranging from 0° C. to reflux temperature preferably at 70-80° C. for a time period of 0.5 hours to 15 hours preferably for 5.0 hours to form acid chloride derivative. This acid chloride derivative can be converted to desired amide compound of formula-I by reaction with ammonia or suitable primary, secondary amine such as methylamine, ethylamine, n-propylamine,isopropylamine,isobutylamine,n-butylamine, Cyclopropyl amine, cyclopentyl amine, cyclohexyl amine. Amine can be any primary or secondary alkyl amines for example, “C₁₋₁₀ alkyl” is intended to include C₁, C₂, C₃, C₄, C₅, C₆, C₇, C₈, C₉, and C₁₀ alkyl groups, in solvents such as dichloromethane, dichloroethane, tetrahydrofuran,acetonitrile,1,4-Dioxane,dimethylformamide, dimethylacetamide or mixture(s) thereof at temperature ranging from 0° C. to reflux temperature preferably at room temperature for a time period of 0.5 hours to 10 hours preferably for 5.0 hours

A compound of formula I-18 can be convertedto a compound of formula I by treating compound of formula I-18 with ammonia or suitable primary, secondary amine such as methylamine, ethylamine, n-propylamine, isopropylamine, isobutylamine, n-butylamine, Cyclopropyl, cyclopentyl, cyclohexyl, using coupling agents such as PyBOP, EDC HCl, DCC, HoBt or coupling agents known to person skilled in the art. Amine can be primary or secondary alkylalkyl amines for example , “C₁₋₁₀ alkyl” is intended to include C₁, C₂, C₃, C₄, C₅, C₆, C₇, C₈, C₉, and C₁₀ alkyl groups, in solvents such as dichloromethane, dichloroethane, tetrahydrofuran, acetonitrile, 1,4-Dioxane, dimethylformamide, dimethylacetamide or mixture(s) thereof at temperature ranging from 0° C. to reflux temperature preferably at room temperature for a time period of 0.5 hours to 15 hours preferably for 10.0 hours.

A compound of formula (I) or its pharmaceutically acceptable salts can be prepared with or without isolation of intermediates.

Isolation of a compound of formula (I) or its pharmaceutically acceptable salts and its intermediates can be carried out by any method known in the art such as cooling, filtration, centrifugation, washing, drying and combination thereof.

Since prodrugs are known to enhance numerous desirable qualities of pharmaceuticals (e.g., solubility, bioavailability, manufacturing, etc.) the compounds of the present invention may be delivered in prodrug form. Thus, the present invention is intended to cover prodrugs of the presently claimed compounds, methods of delivering the same and compositions containing the same. “Prodrugs” are intended to include any covalently bonded carriers that release an active parent drug of the present invention in vivo when such prodrug is administered to a mammalian subject. Prodrugs of the present invention are prepared by modifying functional groups present in the compound in such a way that the modifications are cleaved, either in routine manipulation or in vivo, to give the parent compound.

As used herein, the term “alkyl” is intended to include branched and straight-chain saturated aliphatic hydrocarbon groups and cycloalkyl group having the specified number of carbon atoms. For example, “C₁₋₁₀ alkyl” is intended to include C₁, C₂, C₃, C₄, C₅, C₆, C₇, C₈, C₉, and C₁₀ alkyl groups. Preferred alkyl groups have from 1-6, especially 1-4, carbon atoms. Example alkyl groups include, but are not limited to, methyl (Me),ethyl (Et), propyl (e.g., n-propyl and isopropyl), butyl (e.g., n-butyl, isobutyl, t-butyl), pentyl (e.g., n-pentyl, isopentyl, neopentyl). The said alkyl may be further substituted by alkyl, halogen, amides, esters, acids, cyanide, amines.

The term “cycloalkyl” refers to cyclized alkyl groups, including monocyclic ring systems. C₃₋₁₃ cycloalkyl is intended to include C₃, C₄, C₅, C₆, and C₇cycloalkyl groups. Preferred cycloalkyl groups have from 3-8, especially 3-6, carbon atoms. Example cycloalkyl groups include, but are not limited to, cyclopropyl, cyclobutyl, cyclopentyl, cyclohexyl and the like. The term “protecting group,” as used herein, refers to a labile chemical moiety which is known in the art to protect reactive groups including, without limitation, hydroxyl, amino and thiol groups against undesired reactions during synthetic procedures. Protecting groups are typically used selectively and/or orthogonally to protect sites during reactions at other reactive sites and can then be removed to leave the unprotected group as is or available for further reactions. Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The compounds of formula (I) may exist as a free form (with no ionization) or may form salts which are also within the scope of this invention. Pharmaceutically acceptable (i.e. non-toxic, physiologically acceptable) salts are preferred, although other salts are also useful, e.g., in isolating or purifying the compounds of this invention.

Compounds of this invention may have one or more asymmetric centers. Unless otherwise indicated, all chiral (enantiomeric and diastereomeric) and racemic forms of compounds of the present invention are included in the present invention. Many geometric isomers of olefins, C═N double bonds, and the like can also be present in the compounds, and all such stable isomers are contemplated in the present invention. Cis and trans geometric isomers of the compounds of the present invention are described and may be isolated as a mixture of isomers or as separated isomeric forms. This invention relates to the use of all optical isomers and stereoisomers of the compounds of the present invention, and mixtures thereof, and to all pharmaceutical compositions and methods of treatment that may employ or contain them. The present compounds can be isolated in optically active or racemic forms. It is well known in the art how to prepare optically active forms, such as by resolution of racemic forms or by synthesis from optically active starting materials. All chiral, (enantiomeric and diastereomeric) and racemic forms and all geometric isomeric forms of a structure are intended, unless the specific stereochemistry or isomer form is specifically indicated.

The compounds of this invention include all conformational isomers (e.g., cis and trans isomers). The compounds of the present invention have asymmetric centers and therefore exist in different enantiomeric and diastereomeric forms. This invention relates to the use of all optical isomers and stereoisomers of the compounds of the present invention, and mixtures thereof, and to all pharmaceutical compositions and methods of treatment that may employ or contain them. In this regard, the invention includes both the E and Z configurations. The compounds of formula I may also exist as tautomers. This invention relates to the use of all such tautomers and mixtures thereof. It is to be understood that the invention is not limited merely to one tautomeric form which is illustrated.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. The compounds of formula I may form salts with alkali metals such as sodium, potassium and lithium, with alkaline earth metals such as calcium and magnesium, with organic bases such as dicyclohexylamine, tributylamine, pyridine and amino acids such as arginine, lysine and the like. Such salts can be formed as known to those skilled in the art.

The compounds of formula I may form salts with a variety of organic and inorganic acids. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric, borates and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicyclic, sulfanilic, 2-acetoxybenzoic, fumaric, benzenesulfonic, toluenesulfonic, methanesulfonic, ethane disulfonic, oxalic, isethionic, and the like.

In addition, zwitterions (“inner salts”) may be formed.

The skilled person would appreciate that since the compounds of present invention have more than one basic sites, they have the capacity to form salt with more than one molecule of acid. The present invention embodies mono di or tri salts of the compounds of this disclosure. The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, the disclosure of which is hereby incorporated by reference. Salts can be prepared in the presence or absence of solvents.

“Stable compound”, “Stable isomer(s)” and “stable structure” are meant to indicate a compound that is sufficiently robust to survive isolation to a useful degree of purity from a reaction mixture, and formulation into an efficacious therapeutic agent.

The terms such as “about”, “generally”, “substantially,” and the like are to be construed as modifying a term or value such that it is not an absolute. Such terms will be defined by the circumstances and the terms that they modify as those terms are understood by those skilled in the art. This includes, at the very least, the degree of expected experimental error, technique error and instrument error for a given technique used to measure a value. According to an embodiment the compounds of the present invention, a stereoisomer, tautomer, prodrug or pharmaceutically acceptable salt thereof may be formulated in the suitable form of a composition for pharmaceutical use.

The terms ‘formulation’, ‘composition’, ‘pharmaceutical formulation’ and ‘pharmaceutical composition’ are used interchangeably and refer to preparations which are in such a form as to permit the biological activity of the active ingredients to be effective, and, therefore may be administered to a subject for therapeutic use, wherein the subject is preferably human. ‘Active ingredients’ as used herein refers to the compounds of the present invention.

As understood by the skilled person the suitable form of the composition may be determined by the route of administration of the composition. Therefore the suitable form of the composition may include but is not limited to, injection for intravenous (bolus or infusion), intra-arterial, intraperitoneal, subcutaneous (bolus or infusion), intraventricular, intramuscular, or subarachnoidal route; tablet, capsule, gel, lozenge or liquid for oral ingestion; a solution, suspension or aerosol as sprays for inhalation; gel, spray or cream for topical application; transmucosal composition for administration via oral, nasal or rectal mucosa; by delivery in the form of a transdermal patch, subcutaneous implant, or in the form of a suppository. The compounds may also be formulated in rectal compositions such as suppositories or retention enemas. For buccal administration, the compositions may take the form of tablets or lozenges formulated in conventional manner. The composition may be a vesicular drug delivery system such as, but not limited to, bilosomes, liposomes, niosomes, transferosome, ethosomes, sphingosomes, pharmacosomes, multilamellar vesicles, microspheres and the like.

According to yet another embodiment, the composition of the present invention may comprise the compound of Formula I and a pharmaceutically acceptable excipient. The term ‘excipient’ as used herein refers to inactive or usually inert substances that are added to the formulation which do not affect the therapeutic action of the active ingredient, but serve as a vehicle or medium for the active ingredient. It may be used to provide a desired consistency, to improve stability, and/or to adjust osmolality of the composition.The excipients may be selected from the substances that are known to the skilled person for use in the form of compositions that are dependent on the route of administration. Exemplary excipients include diluents, carriers, binding agents, fillers lubricants, disintegrants, wetting agents, suitable coatings, stabilizers, sterilized water, physiological saline, suitable propellant cocoa butter, glycerides, suspending agents, emulsifying agents, preservatives polymers, solubilizers, cryoprotectants, lyoprotectants, bulking agent/s and/or pharmaceutically acceptable buffers or a mixture thereof. The selection of excipients for preparation of a composition of the present invention is well within the scope and understanding of the skilled person, and suitable excipients are listed in standard references such as Handbook of Pharmaceutical Excipients (Rowe R C, Sheskey P, Quinn M. Pharmaceutical Press; 2009); The Theory And Practice Of Industrial Pharmacy (Lachman, L., Lieberman, H. A., & Kanig, J. L. 1976). The Science and Practice of Pharmacy (Remington J P 2006) and Pharmaceutical Dosage Forms and Drug Delivery Systems (Allen L, Ansel H C 2013 Dec. 23).

A composition of the present invention may be prepared by conventional methods as known to the skilled person.

According to an aspect of the present invention there is provided a method of treatment or prevention of diseases that are treated or prevented by the inhibition of Janus kinases in a subject. Such abnormalities may include but are not limited to proliferative disease, diseases involving impairment of cartilage turnover or diseases involving the anabolic stimulation of chondrocytes, autoimmune diseases, congenital cartilage malformation(s), inflammatory conditions or transplantation rejection.

Proliferative disease refers to a condition such as cancer which is caused by or results in inappropriately high levels of cell division, inappropriately low levels of apoptosis, or both. For example, cancers such as lymphoma, leukaemia, melanoma, ovarian cancer, breast cancer, pancreatic cancer, and lung cancer are examples of proliferative disease. Further JAK2 activating mutations (polycythemiavera, essential thrombocythemia, and myeloid metaplasia with myelofibrosis), psoriasis, restenosis, sclerodermitis or fibrosis are also some of the examples of proliferative disease.

As used herein the term ‘diseases involving impairment of cartilage turnover’ or “diseases involving the anabolic stimulation of chondrocytes” includes conditions such as osteoarthritis, psoriatic arthritis, juvenile rheumatoid arthritis, gouty arthritis, septic or infectious arthritis, reactive arthritis, reflex sympathetic dystrophy, algodystrophy, Tietze syndrome or costal chondritis, fibromyalgia, osteochondritis, neurogenic or neuropathic arthritis, arthropathy, endemic forms of arthritis like osteoarthritis deformansendemica, Mseleni disease and Handigodu disease; degeneration resulting from fibromyalgia, systemic lupus erythematosus, scleroderma and ankylosing spondylitis.

The term ‘congenital cartilage malformation(s)’ includes conditions such as hereditary chondrolysis, chondrodysplasias and pseudochondrodysplasias, in particular, but without limitation, microtia, anotia, metaphyseal chondrodysplasia, and related disorders.

The method of prevention or treatment of the present invention comprises administration of a therapeutically effective amount of a compound of formula I, a stereoisomer, tautomer, prodrug or pharmaceutically acceptable salt thereof to the subject.

The subject may be a mammalian subject. In some embodiments, the subject is human. In particular, the subject may be a human subject suffering from or seeking prevention from a disease related to kinase abnormalities.

According to another aspect of the present invention there is provided a compound of Formula I, a stereoisomer, tautomer, prodrug or pharmaceutically acceptable salt thereof for use in the method of treatment or prevention of Janus kinase mediated disease.

According to yet another aspect of the present invention there is provided a compound of Formula I, a stereoisomer, tautomer, prodrug or pharmaceutically acceptable salt thereof for use as Janus kinase inhibitor.

In an embodiment, the present invention provides a method for the prevention of, or onset of, or progression of Janus kinase related diseases in a subject using a compound of Formula I, a stereoisomer, tautomer, prodrug or pharmaceutically acceptable salt thereof. The invention further provides a method to cure or reduce the effect of diseases caused by Janus kinase abnormalities in a subject using a compound of Formula I or its pharmaceutically acceptable salts.

The treatment or prevention may comprise administering to the subject a therapeutically effective amount of a compound of Formula I, a stereoisomer, tautomer, prodrug or pharmaceutically acceptable salt thereof as such or in a pharmaceutically acceptable form. In some embodiments, the compound is administered at a dose of from 0.01 to 1000 mg/kg, from 0.1 to 100 mg/kg, from 0.5 to 100 mg/kg or from 1 to 50 mg/kg. It will be within the capabilities of the skilled person to determine an amount of the compound to be administered according to the condition to be treated, the chosen route of administration, the actual compound administered, the age, weight, and response of the individual patient, the severity of the patient's symptoms, and the like.

In some embodiments, the compounds of the present invention are administered to the subject enterally, parenterally or topically. In particular the compounds of the present invention may be administered by a suitable route, including but not limited to, injection (including intravenous (bolus or infusion), intra-arterial, intraperitoneal, subcutaneous (bolus or infusion), intraventricular, intramuscular, or subarachnoidal), oral ingestion (e.g. of a tablet, gel, lozenge or liquid), inhalation, topical, via a mucosa (such as the oral, nasal or rectal mucosa), by delivery in the form of a spray, tablet, transdermal patch, subcutaneous implant or in the form of a suppository.

### CELL Free Based Assay (JAK-1, JAK-2, JAK-3 Assay)

The compound of the present invention was dissolved in 300 μl of DMSO to prepare 50 mM stock solution. The stock solution was further diluted in DMSO, in order to perform a cell free assay at SelectScreen®.

The effect of the compound of the present invention on the activity of the JAK kinases has been evaluated using a biochemical assay using purified JAK 1, JAK 2, JAK 3 kinases. In order to perform the assay, Kinase buffer is prepared using 50 mM HEPES pH 6.5, 0.01% BRU-35, 10 mM MgCl₂, 1 mM EGTA, 0.02% NaN3 and Kinase mixture consisting of purified enzymes and fluorophore conjugated-substrates in kinase buffer. Further, test plates were coated with assay mixture comprising the compound of 100 nl of 100X+2.4 μL kinase buffer; 5 μl of Kinase mixture (10 μl of Kinase Reaction consisted of 21.2 ng JAK1 and 2 μM Tyr 06 in kinase buffer); 2.5 μl ATP Solution and assay mixture without compound of present invention was included as Control. The plates were incubated for 1 hr at room temperature and after 1 hr incubation, 5 μL of a 1:128 dilution of development reagent A was added. Plate was incubated for 1 hr at room temperature. Fluorescence was measured on fluorescence plate reader.

The compounds of present invention when tested at concentration ranging from 0.001 μM to 10 μM demonstrated the inhibition of JAK-1 JAK-2 and JAK-3. The percentage inhibition of JAK-1 exhibited by compounds of the present invention was between 20% to 99%, particularly at concentrations from 0.1 to μM 10 μM. The percentage inhibition of JAK-2 was a little lower as compared to JAK-1. At a concentration range from 0.1 μM to 10 the compounds inhibited JAK-2 by 10% to 95%. Percentage inhibition of JAK-3 by the compounds of present invention at a concentration from 0.1 μM to 10 μM was 8% to 99%. Compounds with higher alkyl groups for example alkyl with C₃ to C₁₀ carbons on the amide nitrogen exhibited higher percentage inhibition even at lower concentration ranges such as 0.001 μM to 0.03 μM. For example, compounds with a propyl or isopropyl group on this nitrogen inhibited JAK-1 by 5% to 25% at a concentration of 0.001 μM to 0.03 μM. and JAK-3 by 25% ata concentration 0.03 μM.

### PREPARATIONS AND EXAMPLES

It is to be understood that the following examples do not limit the invention and are only meant to suggest a method of practicing the invention. Persons skilled in the art will recognize that the chemical reactions described may be readily adapted to prepare other compounds of formula I, and alternative methods for preparing the compounds of formula I are within the scope of this invention. For example, the synthesis of non-exemplified compounds according to the invention may be successfully performed by modifications apparent to those skilled in the art, e.g., by appropriately protecting interfering groups, by utilizing other suitable reagents known in the art other than those described, and/or by making routine modifications of reaction conditions. Alternatively, other reactions disclosed herein or known in the art will be recognized as having applicability for preparing other compounds of the invention.

### Example 1: N,1-dimethyl-1,6-dihydroimidazo[4,5-d]pyrrolo[2,3-b]pyridine-8-carboxamide

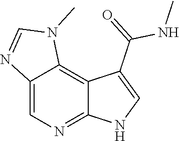 [SMILES: CNC(=O)c1c[nH]c2ncc3ncn(C)c3c12]

### Step A: 2-chloro-N-methyl-5-nitropyridin-4-amine

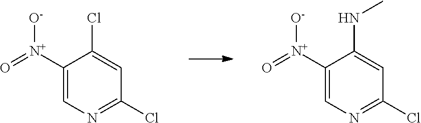

A solution of 2,4-dichloro-5-nitropyridine (15 mmol) in methyl amine (2M solution in toluene, 15 mL) was stirred at 40-50° C. for 18 h. The reaction mixture was concentrated by evaporation in vacuo, then the residue was isolated by filtration and purified by hexane wash (3×30 mL), to provide the title compound as solid. (Yield 68%)

### Step B: 6-chloro-N-4-methylpyridine-3,4-diamine

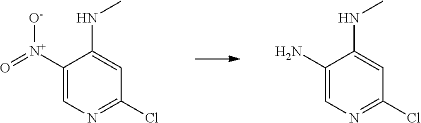

A suspension of 2-chloro-N-methyl-5-nitropyridin-4-amine (15 mmol) in 100 ml ethanol was treated with Pd/C (10% Pd) and hydrogenated for 12 h under atmospheric pressure. The reaction mixture was filtered through a plug of celite and the filtrate was concentrated under vacuum and purified by preparative HPLC to yield the title compound. (Yield 72%)

### Step B1: 6-chloro-N-4-methylpyridine-3,4-diamine

To a solution of 2-chloro-N-methyl-5-nitropyridin-4-amine (25 mmol) in EtOAc (100 mL) was added Raney nickel (10% Pd) followed by stirring under hydrogen at RT for 2 h. The reaction mixture was filtered and the filtrate concentrated under reduced pressure to afford (80%) of crude 6-chloro-N-4-methylpyridine-3,4-diamine (Yield 74%).

### Step C: 6-chloro-1-methyl-1H-imidazo[4,5-c]pyridine

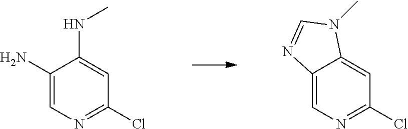

A suspension of 2-chloro-N-methyl-5-nitropyridin-4-amine (25 mmol) in 100 ml ethanol was treated with Pd/C (10% Pd) and hydrogenated for 12 h under atmospheric pressure. The reaction mixture was filtered through a plug of celite and the filtrate was concentrated under vacuum to give 6-chloro-N-4-methylpyridine-3,4-diamine. The resulting oil was treated with (50 mmol) diethoxymethyl acetate and stirred for 4 h at room temperature and for one hour at 90° C. The reaction mixture was allowed to cool down to room temperature, 50 ml dichloromethane was added and the organic layer was washed with water (4×20 ml). The combined organics layers were concentrated to a volume of 10 ml and purified by preparative HPLC to yield the title compound. (Yield 70%)

### Step D: 1-methyl-1H-imidazo[4,5-c]pyridin-6-amine

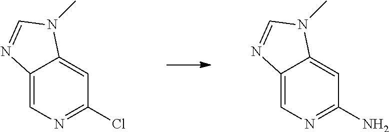

6-chloro-1-methyl-1H-imidazo[4,5-c]pyridine compound (6.5 g) and 20 ml of 28% aqueous ammonia were placed in a 50 ml autoclave, and the mixture was reacted for 24 hours at 100° C., and further for 5 hours at 125° C. (inner pressure: about 2 atms.). After completion of the reaction, the reaction product was allowed to cool to obtain crystals. The thus obtained crystals were then washed with water and dried to obtain the title compound (Yield 74%).

### Step D: 1-methyl-1H-imidazo[4,5-c]pyridin-6-amine

To 6-chloro-1-methyl-1H-imidazo[4,5-c]pyridine (6.5 mmol) in toluene under nitrogen was added racemic BINAP (0.4 mmol), Pd2(dba)3 (0.13 mmol) and sodium tert-butoxide (9.1 mmol), Benzophenoneimine (7.81 mmol) was added and the mixture was heated to 80° C. for 3 h and cooled to room temperature. The reaction mixture was diluted with ether, filtered through Celite, and washed with ether. The filtrate was concentrated and the residue was taken up in methanol (90 ml) and treated with hydroxylamine (19.5 mmol). The mixture was stirred at ambient temperature for 18 h and concentrated. The residue was purified by column chromatography (95-100% ethyl acetate/hexanes) to afford the title compound. (84% yield).

### Step E: 7-iodo-1-methyl-1H-imidazo[4,5-c]pyridin-6-amine

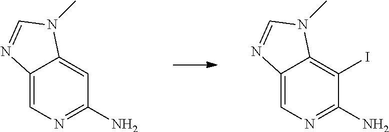

In a round bottom flask, 1-methyl-1H-imidazo[4,5-c]pyridin-6-amine (19.08 mmol) was dissolved in MeCN (200 mL) and cooled to 0° C. N-Iodosuccinimide (20.03 mmol) was dissolved in the remaining MeCN (50 mL) and added dropwise to the reaction mixture over 40 min. The reaction mixture was stirred at 0° C. for an additional 10 min and was quenched with 2M sodium hydrogensulfite (125 mL). Stirring and temperature were maintained for 50 min. The mixture was transferred to a separatory funnel. The aqueous layer was extracted with MDC (3×100 mL).The combined organic layers were washed with water and brine, dried over sodium sulfate, filtered and concentrated in vacuo. The crude residue was purified by chromatography eluting with 20-100% ethylacetate/Heptane to provide the title compound (72% yield).

### Step F: 7-ethynyl-1-methyl-1H-imidazo[4,5-c]pyridin-6-amine

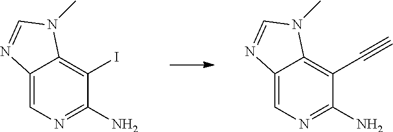

Trimethylsilylacetylene (700 mmol) in THF (150 mL) solution was added via cannula to a cooled (0-5° C.), degassed mixture of 7-iodo-l-methyl-1H-imidazo[4,5-c]pyridin-6-amine (465 mmol), bis(triphenylphosphine) dichloropalladium(0) (23.2 mmol), copper (I) iodide (27.9 mmol) and triethylamine (1.4 mol) in THF (1.25 L). The mixture was stirred at 0-5° C. for 30 minutes then for a further 30 minutes at ambient temperature. The solid was removed by filtration and the cake washed with THF. The filtrate was diluted with ethyl acetate and extracted with 2M hydrochloric acid. The combined acid extract was washed with diethyl ether and then made basic by careful addition of potassium carbonate then extracted with diethyl ether. The combined organic layer was dried (Na₂SO₄), filtered and evaporated. The resultingresidue was dissolved in tetrahydrofuran solution (300 mL). Tetrabutylammonium fluoride (20 mmol in a 1 M tetrahydrofuran solution) was added in reaction mass and stirred for 2-3 hrs at room temperature. Water was added to the reaction solution, which was then extracted four times with ethyl acetate. The organic layer was dried over anhydrous sodium sulfate, and the solvent was evaporated under a reduced pressure. The residue was purified by silica gel chromatography (heptane:ethyl acetate=1:1, then 1:2), which gave the titled compound (72%).

### Step G: 1-methyl-1,6-dihydroimidazo[4,5-d]pyrrolo[2,3-b]pyridine

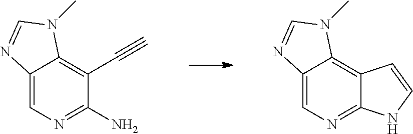

150 g of DMF was added to a reactor, 70 g of potassium t-butoxide was slowly added, the reaction mixture was stirred to 60-70° C., and 50 g of 7-ethynyl-1-methyl-1H-imidazo[4,5-c]pyridin-6-amine was slowly added. The temperature control was not higher than 80° C., and the reaction was completed by incubation at 80-85° C. for 3 hrs, TLC monitoring completion of the reaction (PE/DCM=1/1), cooled after completion of the reaction, the reaction system was slowly added to 400 g of ice water, cooled to 10° C., was stirred for 2 hrs, filtered off with suction to give a solid crude wet weight of about 75 g. The wet product was added to a 500 mL reaction vessel, 300 g of ethyl acetate (EA) was added, 5 g of activated carbon was added, and the mixture was heated under reflux for 30 minutes, and then suction filtered. The filter cake was washed with an appropriate amount of EA, and then the filtrate and the washing liquid were combined, and the EA was evaporated to about 250 g under reduced pressure, cooling to 0-5° C. for 2 hrs, suction filtration, filter cake washed with appropriate amount of cold EA, drying at 60° C. to obtain a solid product title compound (80%).

### Step H: 8-bromo-1-methyl-1,6-dihydroimidazo[4,5-d]pyrrolo[2,3-b]pyridine

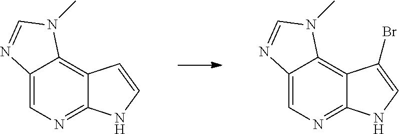

1-methyl-1,6-dihydroimidazo[4,5-d]pyrrolo[2,3-b]pyridine (0.9 mmol) was dissolved in THF (25 mL) at room temperature and to the resulting solution was added N-bromosuccinimide (1.08 mmol). The resulting suspension was stirred at room temperature for 14 hours, then quenched with aqueous saturate sodium thiosulfate solution (10 mL). The reaction was concentrated in vacuo, and the resulting residue was diluted with ethyl acetate (50 mL). The aqueous layer was extracted with ethyl acetate (50 mL) and the combined organic layers were washed with aqueous 1N sodium bicarbonate solution (10 mL) and brine (10 mL), then dried over magnesium sulfate, filtered and concentrated in vacuo to provide the title compound (85%), which was used further with or without purification.

### Step I: Ethyl 1-methyl-1,6-dihydroimidazo[4,5-d]pyrrolo[2,3-b]pyridine-8-carboxylate

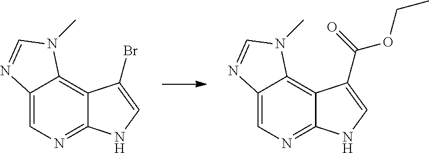 [SMILES: Cn1cnc2cnc3[nH]cc(Br)c3c21 ; CCOC(=O)c1c[nH]c2ncc3ncn(C)c3c12]

8-bromo-1-methyl-1,6-dihydroimidazo[4,5-d]pyrrolo[2,3-b]pyridine (173 mmol) was added in dry tetrahydrofuran (500 mL) at −78° C. and n-butyl lithium (2.5 M solution in hexane, 487 mmol) was added over a period of 2 hours. The reaction mixture was stirred for another 30 minutes at −78° C. Ethyl chloroformate (186 mmol) was added over 30 minutes and the reaction mixture was stirred for 2 hours at −60° C. The temperature was slowly increased to 30° C. and the mixture was allowed to stir for 12 hours at 30° C. The progress of the reaction was monitored by TLC. The reaction mixture was then quenched with saturated solution of ammonium chloride (150 mL) at 0° C. and the reaction mixture was extracted with ethyl acetate (3×300 mL). The combined organic layers were washed with water, dried over 10 anhydrous sodium sulfate (50 g), filtered and concentrated under reduced pressure to afford a crude reaction mixture. The residue was purified by chromatography to provide the title compound (52%).

1H NMR (400 MHz, DMSO-d6) δ: 1.26 (t, 3 H), 3.23 (s, 3 H), 4.14 (q, 2 H), 7.90 (s, 1H), 8.42 (s, 1 H), 8.95 (s, 1 H), 12.84 (br s, 1H).

### Step J: N,1-dimethyl-1,6-dihydroimidazo[4,5-d]pyrrolo[2,3-b]pyridine-8-carboxamide

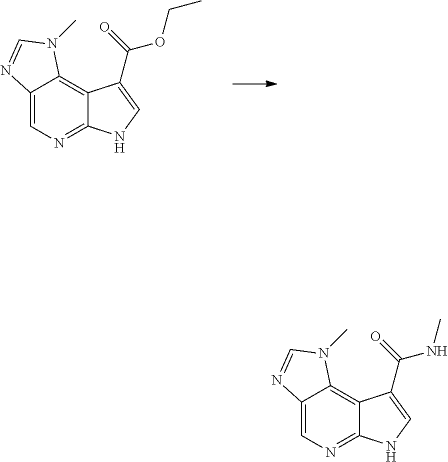 [SMILES: CCOC(=O)c1c[nH]c2ncc3ncn(C)c3c12 ; CNC(=O)c1c[nH]c2ncc3ncn(C)c3c12]

A solution of trimethylaluminium (2 M in toluene, 1.2 mmol) was added dropwise (exothermic) to a solution of methylamine (2 M in toluene, 1.2 mmol) in dioxane (7.5 mL) and the resulting mixture was stirred at room temperature for 1 h. Then a solution of Ethyl 1-methyl-1,6-dihydroimidazo[4,5-d]pyrrolo[2,3-b]pyridine-8-carboxylate (0.3 mmol) in dioxane (4 mL) was added. The resulting mixture was then heated at 85-95° C. for 3 h and then cooled to room temperature and then poured into water and extracted with MDC which was then washed with brine, dried over sodium sulfate and evaporated. Purification by chromatography (SiO2, MDC:Me0H=90:10) afforded the title compound as a white solid. (72%).

1H NMR (400 MHz, CD3OD) δ: 2.96 (s, 3 H), 4.15 (s, 3 H), 7.74 (s, 1H), 8.16 (s, 1H), 8.64 (s, 1H).

### Step J-1: Alternate method for preparation of N,1-dimethyl-1,6-dihydroimidazo[4,5-d]pyrrolo[2,3-b]pyridine-8-carboxamide

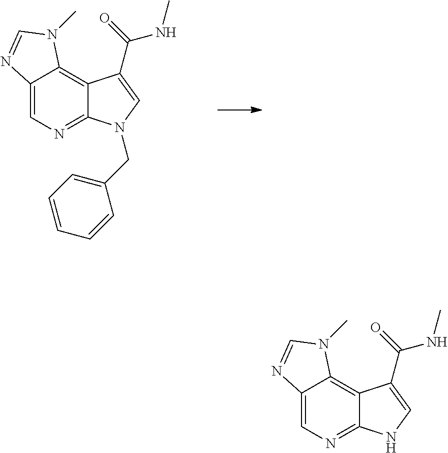 [SMILES: CNC(=O)c1cn(Cc2ccccc2)c2ncc3ncn(C)c3c12 ; CNC(=O)c1c[nH]c2ncc3ncn(C)c3c12]

In a round bottom flask, ammonia gas was condensed to liquid ammonia (10 -50 vol) at −60° C. to −80° C. Sodium metal (25 mmol) was added portionwise in above reaction mass.

After stirring to complete dissolution, dark blue colouration was observed. 6-benzyl-N,1-dimethyl-1,6-dihydroimidazo[4,5-d]pyrrolo[2,3-b]pyridine-8-carboxamide (5 mmol) was charged in a reaction mass at −60° C. to −80° C., the reaction mixture was stirred at −60° C. to −80° C. for an additional 60 min. The reaction mixture was heated to −30° C. and saturated ammonium chloride solution (25 vol) was added slowly and stirred for 30 min. the reaction mixture was heated to room temperature and stirred for 1.0 hr. The reaction mass was extracted with 5% methanol in MDC and the combined organic layers then dried over magnesium sulfate. The crude residue was purified by chromatography eluting with 2-30% Methanol/MDC to provide the title compound (35% yield).

1H NMR (400 MHz, CD3OD) δ: 2.96 (s, 3 H), 4.15 (s, 3 H), 7.74 (s, 1 H), 8.16 (s, 1H), 8.64 (s, 1H).

### Example 2: N-ethyl-1-methyl-1,6-dihydroimidazo[4,5-d]pyrrolo[2,3-b]pyridine-8-carboxamide

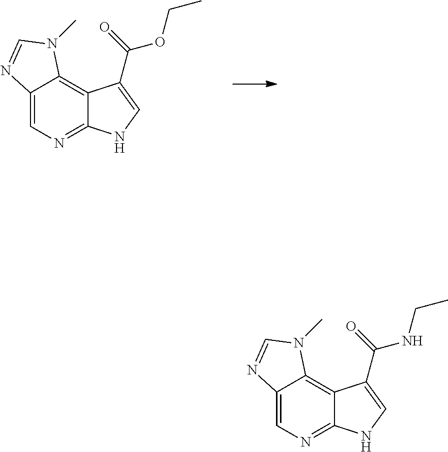 [SMILES: CCOC(=O)c1c[nH]c2ncc3ncn(C)c3c12 ; CCNC(=O)c1c[nH]c2ncc3ncn(C)c3c12]

A solution of trimethylaluminium (2 M in toluene, 1.2 mmol) was added dropwise (exothermic) to a solution of Ethylamine (2 M in toluene, 1.2 mmol) in dioxane (7.5 mL) and the resulting mixture was stirred at room temperature for 1 h. Then a solution of Ethyl 1-methyl-1,6-dihydroimidazo[4,5-d]pyrrolo[2,3-b]pyridine-8-carboxylate (0.3 mmol) in dioxane (4 mL) was added. The resulting mixture was then heated at 85-95° C. for 3 h and then cooled to room temperature and then poured into water and extracted with MDC which was then washed with brine, dried over sodium sulfate and evaporated. Purification by chromatography (SiO2, MDC:MeOH=90:10) afforded the title compound as a white solid. (72%).

1H NMR (400 MHz, CD3OD) δ: 1.27 (t, 3 H), 3.44 (q, 2 H), 4.14 (s, 3H), 7.73 (s, 1 H), 8.13 (s, 1H), 8.63 (s, 1H).

### Example 3: 1-methyl-N-propyl-1,6-dihydroimidazo[4,5-d]pyrrolo [2,3-b]pyridine-8-carboxamide

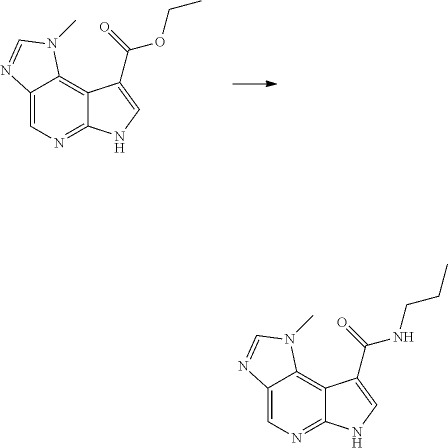 [SMILES: CCOC(=O)c1c[nH]c2ncc3ncn(C)c3c12 ; CCCNC(=O)c1c[nH]c2ncc3ncn(C)c3c12]

A solution of trimethylaluminium (2 M in toluene, 1.2 mmol) was added dropwise (exothermic) to a solution of n-propyl amine (2 M in toluene, 1.2 mmol) in dioxane (7.5 mL) and the resulting mixture was stirred at room temperature for 1 h. Then a solution of Ethyl 1-methyl-1,6-dihydroimidazo[4,5-d]pyrrolo[2,3-b]pyridine-8-carboxylate (0.3 mmol) in dioxane (4 mL) was added. The resulting mixture was then heated at 85-95° C. for 3 h and then cooled to room temperature and then poured into water and extracted with MDC which was then washed with brine, dried over sodium sulfate and evaporated. Purification by chromatography (SiO2, MDC:MeOH=90:10) afforded the title compound as a white solid. (72%).

1H NMR (400 MHz, CD3OD) δ: 1.03 (t, 3 H), 1.71-1.64 (m, 2 H), 3.37 (t, 2 H), 4.14 (s, 3H), 7.73 (s, 1 H), 8.12 (s, 1H), 8.63 (s, 1H).

### Example 4: N-methyl-1,6-dihydroimidazo[4,5-d]pyrrolo[2,3-b]pyridine-8-carboxamide

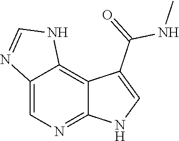 [SMILES: CNC(=O)c1c[nH]c2ncc3nc[nH]c3c12]

### Step A: 2-chloro-5-nitropyridin-4-amine

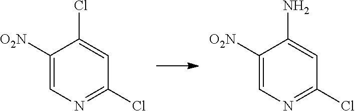 [SMILES: O=[N+]([O-])c1cnc(Cl)cc1Cl ; Nc1cc(Cl)ncc1[N+](=O)[O-]]

A solution of 2,4-dichloro-5-nitropyridine (15 mmol) in methanolic ammonia (15 mL) was stirred at 25-28° C. for 18 h. The reaction mixture was concentrated by evaporation in vacuo, 5 then the residue was isolated by filtration and purified by hexane wash (3×30 mL), to provide the title compound as a solid. (Yield 86%)

### Step B: 6-chloropyridine-3,4-diamine

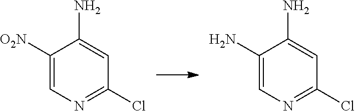 [SMILES: Nc1cc(Cl)ncc1[N+](=O)[O-] ; Nc1cnc(Cl)cc1N]

A suspension of 2-chloro-5-nitropyridin-4-amine (15 mmol) in 100 ml ethanol was treated with Pd/C (10% Pd) and hydrogenated for 12 h under atmospheric pressure. The reaction mixture was filtered through a plug of celite and the filtrate was concentrated under vacuum and purified by preparative HPLC to yield the title compound. (Yield 72%)

### Step B1: 6-chloropyridine-3,4-diamine

To a solution of 2-chloro-5-nitropyridin-4-amine (25 mmol) in EtOAc (100 mL) was added Raney nickel (10% Pd) followed by stirring under hydrogen at RT for 2 h. The reaction mixture was filtered and the filtrate concentrated under reduced pressure to afford (80%) of crude 6-chloropyridine-3,4-diamine. (Yield 74%)

### Step C: 6-chloro-1H-imidazo[4,5-c]pyridine

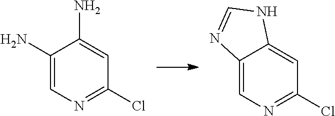 [SMILES: Nc1cnc(Cl)cc1N ; Clc1cc2[nH]cnc2cn1]

A suspension of 6-chloropyridine-3,4-diamine (25 mmol) in 100 ml ethanol was treated with Pd/C (10% Pd) and hydrogenated for 12 h under atmospheric pressure. The reaction mixture was filtered through a plug of celite and the filtrate was concentrated under vacuum to give (Example A-3). The resulting oil was treated with (50 mmol) diethoxymethyl acetate and stirred for 4 h at room temperature and for one hour at 90° C. The reaction mixture was allowed to cool down to room temperature, 50 ml dichloromethane was added and the organic layer was washed with water (4×20 ml). The combined organics layers were concentrated to a volume of 10 ml and purified by preparative HPLC to yield the title compound. (Yield 70%)

### Step D: 1H-imidazo[4,5-c]pyridin-6-amine

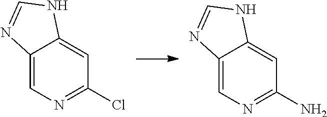 [SMILES: Clc1cc2[nH]cnc2cn1 ; Nc1cc2[nH]cnc2cn1]

6.5 g of 6-chloro-1H-imidazo[4,5-c]pyridine compound and 20 ml of 28% aqueous ammonia were placed in a 50 ml autoclave, and the mixture was reacted for 24 hours at 100° C., and further for 5 hours at 125° C. (inner pressure: about 2 atms.). After completion of the reaction, the reaction product was allowed to cool to obtain crystals. The thus obtained crystals were then washed with water and dried to obtain the title compound (Yield 74%).

### Step D1: 1H-imidazo[4,5-c]pyridin-6-amine

To 6-chloro-1H-imidazo[4,5-c]pyridine (6.5 mmol) in toluene under nitrogen was added racemic BINAP (0.4 mmol), Pd2(dba)3 (0.13 mmol) and sodium tert-butoxide (9.1 mmol), Benzophenoneimine (7.81 mmol) was added and the mixture was heated to 80° C. for 3 h and cooled to room temperature. The reaction mixture was diluted with ether, filtered through Celite, and washed with ether. The filtrate was concentrated and the residue was taken up in methanol (90 ml) and treated with hydroxylamine (19.5 mmol). The mixture was stirred at ambient temperature for 18 h and concentrated. The residue was purified by column chromatography (95-100% ethyl acetate/hexanes) to afford the title compound. (84% yield).

### Step E: 7-iodo-1H-imidazo[4,5-c]pyridin-6-amine

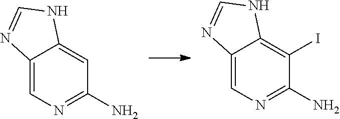 [SMILES: Nc1cc2[nH]cnc2cn1 ; Nc1ncc2nc[nH]c2c1I]

In a round bottom flask, 1H-imidazo[4,5-c]pyridin-6-amine (19.08 mmol) was dissolved in MeCN (200 mL) and cooled to 0° C. N-Iodosuccinimide (20.03 mmol) was dissolved in the remaining MeCN (50 mL) and added dropwise to the reaction mixture over 40 min. The reaction mixture was stirred at 0° C. for an additional 10 min and was quenched with 2M sodium hydrogensulfite (125 mL). Stirring and temperature were maintained for 50 min. The mixture was transferred to a separatory funnel. The aqueous layer was extracted with MDC (3×100 mL).The combined organic layers were washed with water and brine, dried over sodium sulfate, filtered and concentrated in vacuo. The crude residue was purified by chromatography eluting with 20-100% ethylacetate/Heptane to provide title compound (72% yield).

### Step F: 7-ethynyl-1H-imidazo[4,5-c]pyridin-6-amine

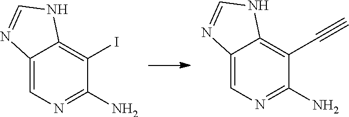

Trimethylsilylacetylene (700 mmol) THF (150 mL) solution was added via cannula to a cooled (0-5° C.), degassed mixture of 7-iodo-1H-imidazo[4,5-c]pyridin-6-amine (465 mmol), bis(triphenylphosphine) dichloropalladium(0) (23.2 mmol), copper (I) iodide (27.9 mmol) and triethylamine (1.4 mol) in THF (1.25 L). The mixture was stirred at 0-5° C. for 30 minutes then for a further 30 minutes at ambient temperature. The solid was removed by filtration and the cake washed with THF. The filtrate was diluted with ethyl acetate and extracted with 2M hydrochloric acid. The combined acid extract was washed with diethyl ether and then made basic by careful addition of potassium carbonate then extracted with diethyl ether. The combined organic layer was dried (Na₂SO₄), filtered and evaporated. The resulting residue was dissolved in tetrahydrofuran solution (300 mL). Tetrabutylammonium fluoride (20 mmol in a 1 M tetrahydrofuran solution) was added in reaction mass and stirred for 2-3 hrs at room temperature. Water was added to the reaction solution, which was then extracted four times with ethyl acetate. The organic layer was dried over anhydrous sodium sulfate, and the solvent was evaporated under a reduced pressure. The residue was purified by silica gel chromatography (heptane:ethyl acetate=1:1, then 1:2), which gave the title compound (68%)

### Step G: 1,6-dihydroimidazo[4,5-d]pyrrolo[2,3-b]pyridine

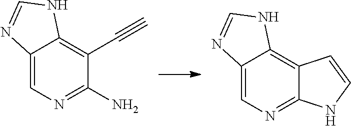

150 g of DMF was added to a reactor, 70 g of potassium t-butoxide was slowly added, stirred to 60-70° C., and 50 g of 7-ethynyl-1H-imidazo[4,5-c]pyridin-6-amine was slowly added. The temperature control was not higher than 80° C. , plus the reaction was completed by incubation at 80-85° C. for 3 hrs, TLC monitoring completion of the reaction (PE/DCM=1/1), cooled after completion of the reaction, the reaction system was slowly added to 400 g of ice water, cooled to 10° C., was stirred for 2 hrs, filtered off with suction to give a solid crude wet weight of about 75 g. The wet product was added to a 500 mL reaction vessel, 300 g of ethyl acetate (EA) was added, 5 g of activated carbon was added, and the mixture was heated under reflux for 30 minutes, and then suction filtered. The filter cake was washed with an appropriate amount of EA, and then the filtrate and the washing liquid were combined, and the EA was evaporated to about 250 g under reduced pressure, cooling to 0-5° C. for 2 hrs, suction filtration, filter cake washed with appropriate amount of cold EA, drying at 60° C. to obtain a solid product title compound (80%).

### Step H: 8-bromo-1,6-dihydroimidazo[4,5-d]pyrrolo[2,3-b]pyridine

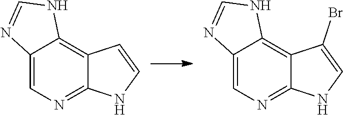 [SMILES: c1cc2c(ncc3nc[nH]c32)[nH]1 ; Brc1c[nH]c2ncc3nc[nH]c3c12]

1,6-dihydroimidazo[4,5-d]pyrrolo[2,3-b]pyridine (0.9 mmol) was dissolved in THF (25 mL) at room temperature and to the resulting solution was added N-bromosuccinimide (1.08 mmol). The resulting suspension was stirred at room temperature for 14 hours, then quenched with aqueous saturated sodium thiosulfate solution (10 mL). The reaction was concentrated in vacuo, and the resulting residue was diluted with ethyl acetate (50 mL). The aqueous layer was extracted with ethyl acetate (50 mL) and the combined organic layers were washed with aqueous 1N sodium bicarbonate solution (10 mL) and brine (10 mL), then dried over magnesium sulfate, filtered and concentrated in vacuo to provide the title compound (85%), which was used further with or without purification.

### Step I: Ethyl 1,6-dihydroimidazo[4,5-d]pyrrolo[2,3-b]pyridine-8-carboxylate

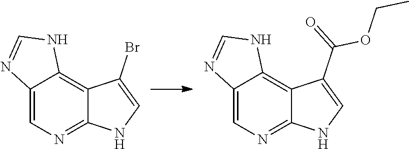 [SMILES: Brc1c[nH]c2ncc3nc[nH]c3c12 ; CCOC(=O)c1c[nH]c2ncc3nc[nH]c3c12]

8-bromo-1,6-dihydroimidazo[4,5-d]pyrrolo[2,3-b]pyridine (173 mmol) was added in dry tetrahydrofuran (500 mL) at −78° C. and n-butyl lithium (2.5 M solution in hexane, 487 mmol) was added over a period of 2 hours. The reaction mixture was stirred for another 30 minutes at −78° C. Ethyl chloroformate (186 mmol) was added over 30 minutes and the reaction mixture was stirred for 2 hours at −60° C. The temperature was slowly increased to 3° C. and mixture was allowed to stir for 12 hours at 30° C. The progress of the reaction was monitored by TLC. The reaction mixture was then quenched with a saturated solution of ammonium chloride (150 mL) at 0° C. and the reaction mixture was extracted with ethyl acetate (3×300 mL). The combined organic layers were washed with water, dried over anhydrous sodium sulfate (50 g), filtered and concentrated under reduced pressure to afford a crude reaction mixture. The residue was purified by chromatography to provide the title compound (52%).

1H NMR (400 MHz, DMSO-d6) δ: 1.26 (t, 3 H), 3.23 (s, 3 H), 4.14 (q, 2 H), 7.90 (s, 1H), 8.42 (s, 1 H), 8.95 (s, 1 H), 12.84 (br s, 1H).

### Step J: N,1-dimethyl-1,6-dihydroimidazo[4,5-d]pyrrolo[2,3-b]pyridine-8-carboxamide

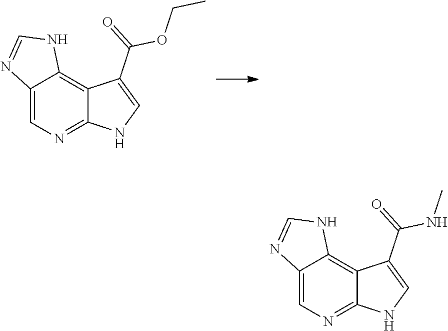 [SMILES: CCOC(=O)c1c[nH]c2ncc3nc[nH]c3c12 ; CNC(=O)c1c[nH]c2ncc3nc[nH]c3c12]

A solution of trimethylaluminium (2 M in toluene, 1.2 mmol) was added dropwise (exothermic) to a solution of methylamine (2 M in toluene, 1.2 mmol) in dioxane (7.5 mL) and the resulting mixture was stirred at room temperature for 1 h. Then a solution of Ethyl 1,6-dihydroimidazo[4,5-d]pyrrolo[2,3-b]pyridine-8-carboxylate (0.3 mmol) in dioxane (4 mL) was added. The resulting mixture was then heated at 85-95° C. for 3 h and then cooled to room temperature and then poured into water and extracted with MDC which was then washed with brine, dried over sodium sulfate and evaporated. Purification by chromatography (SiO2, MDC:MeOH=90:10) afforded the title compound as a white solid. (72%).

1H NMR (400 MHz, CD3OD) δ: 3.04 (s, 3 H), 8.02 (s, 1 H), 8.35 (s, 1H), 8.66 (s, 1H).

### Example 5: N-ethyl-1,6-dihydroimidazo[4,5-d]pyrrolo[2,3-b]pyridine-8-carboxamide

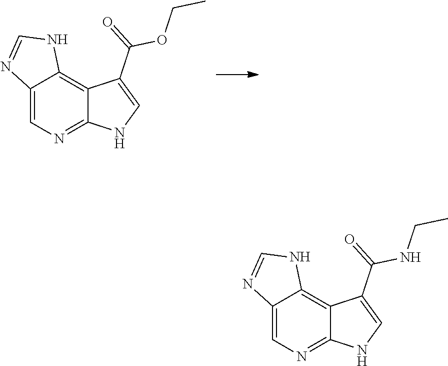 [SMILES: CCOC(=O)c1c[nH]c2ncc3nc[nH]c3c12 ; CCNC(=O)c1c[nH]c2ncc3nc[nH]c3c12]

A solution of trimethylaluminium (2 M in toluene, 1.2 mmol) was added dropwise (exothermic) to a solution of ethylamine (2 M in toluene, 1.2 mmol) in dioxane (7.5 mL) and the resulting mixture was stirred at room temperature for 1 h. Then a solution of Ethyl 1,6-dihydroimidazo[4,5-d]pyrrolo[2,3-b]pyridine-8-carboxylate (0.3 mmol) in dioxane (4 mL) was added. The resulting mixture was then heated at 85-95° C. for 3 h and then cooled to room temperature and then poured into water and extracted with MDC which was then washed with brine, dried over sodium sulfate and evaporated. Purification by chromatography (SiO2, MDC:MeOH=90:10) afforded the title compound as a white solid. (72%).

1H NMR (400 MHz, DMSOd6) δ: 1.19 (t, 3 H), 3.39 (q, 2 H), 7.98 (s, 1 H), 8.29 (s, 1H), 8.62 (s, 1H), 9.78 (bs, 1H), 12.12 (bs, 1H).

### Example 6: N-(propan-2-yl)-1,6-dihydroimidazo[4,5-d]pyrrolo[2,3-b]pyridine-8-carboxamide

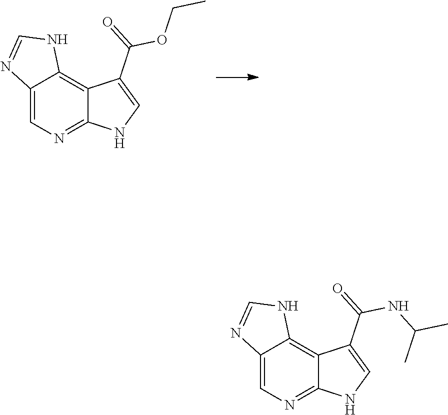 [SMILES: CCOC(=O)c1c[nH]c2ncc3nc[nH]c3c12 ; CC(C)NC(=O)c1c[nH]c2ncc3nc[nH]c3c12]

A solution of trimethylaluminium (2 M in toluene, 1.2 mmol) was added dropwise (exothermic) to a solution of isopropylamine(1.2 mmol) in dioxane (7.5 mL) and the resulting mixture was stirred at room temperature for 1 h. Then a solution of Ethyl 1,6-dihydroimidazo[4,5-d]pyrrolo[2,3-b]pyridine-8-carboxylate (0.3 mmol) in dioxane (4 mL) was added. The resulting mixture was then heated at 85-95° C. for 3 h and then cooled to room temperature and then poured into water and extracted with MDC which was then washed with brine, dried over sodium sulfate and evaporated. Purification by chromatography (SiO2, MDC:MeOH=90:10) afforded the title compound as a white solid. (72%).

1H NMR (400 MHz, CD3OD) δ: 1.25 (d, 6 H), 4.23 (m, 1 H), 7.99 (s, 1 H), 8.34 (s, 1H), 8.57 (s, 1H).

### Example 7: N-(propan-2-yl)-1,6-dihydroimidazo[4,5-d]pyrrolo[2,3-b]pyridine-8-carboxamide hydrochloride

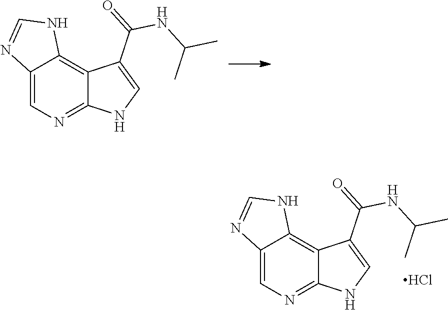 [SMILES: CC(C)NC(=O)c1c[nH]c2ncc3nc[nH]c3c12 ; CC(C)NC(=O)c1c[nH]c2ncc3nc[nH]c3c12 ; Cl]

Ethanolic hydrochloride solution was added to the solution of N-(propan-2-yl)-1,6-dihydroimidazo[4,5-d]pyrrolo[2,3-b]pyridine-8-carboxamide (10 mmol) in 50 mL ethanol. The reaction was stirred for 3-4 hrs at ambient temperature. Reaction mass was concentrated under reduced pressure. 10 ml ethanol was added to the residue, stirred and reaction mass was concentrated under reduced pressure. Obtained solid was dried under vacuum to afford white to off while solid of the title compound (Yield=95%).

1H NMR (400 MHz, DMSO-D6) δ: 1.25 (d, 6 H), 4.23 (m, 1 H), 7.99 (s, 1 H), 8.34 (s, 1H), 8.57 (s, 1H), δ8.7 (bs1H), 11.2 (br. S, 1H), 12.9 (br. S, 2H).

### Preparation of an Intermediate:6-benzyl-N,1-dimethyl-1,6-dihydroimidazo[4,5-d]pyrrolo[2,3-b]pyridine-8-carboxamide

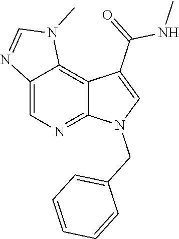 [SMILES: CNC(=O)c1cn(Cc2ccccc2)c2ncc3ncn(C)c3c12]

### Step A: 6-benzyl-1-methyl-1,6-dihydroimidazo[4,5-d]pyrrolo[2,3-b]pyridine

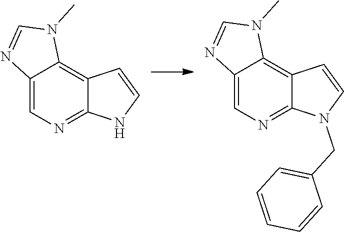 [SMILES: Cn1cnc2cnc3[nH]ccc3c21 ; Cn1cnc2cnc3c(ccn3Cc3ccccc3)c21]

In a round bottom flask, sodium hydride (0.3 mole) was added in a DMF (5 vol) solvent, 1-methyl-1,6-dihydroimidazo[4,5-d]pyrrolo[2,3-b]pyridine (0.1 mole) was slowly added at 5-15° C. to a flask, the resulting suspension was stirred at room temperature for 1 hour, then benzyl bromide (0.12 mole) was slowly added at 5-15° C. The reaction mass was warmed to room temperature and stirred for 1-3 hours. The reaction was monitored on TLC. The reaction mass was cooled after completion of the reaction, methanol (1 vol) was added in the reaction mass at 5-15° C. and stirred for 10 min. Ammonium chloride (25 vol) solution was added in the reaction mass and stirred for 30 min. The reaction mass was extracted with ethyl acetate (3*5 vol). Combined organic layers were washed with water (3* 5 vol) and brine (1 vol), then dried over magnesium sulfate, filtered and concentrated in vacuo to provide the title compound (86%).

### Step B: 6-benzyl-8-bromo-1-methyl-1,6-dihydroimidazo[4,5-d]pyrrolo[2,3-b]pyridine

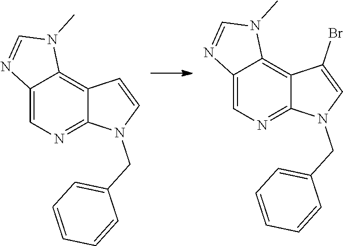 [SMILES: Cn1cnc2cnc3c(ccn3Cc3ccccc3)c21 ; Cn1cnc2cnc3c(c(Br)cn3Cc3ccccc3)c21]

6-benzyl-1-methyl-1,6-dihydroimidazo[4,5-d]pyrrolo[2,3-b]pyridine (1 mmol) was dissolved in THF (25 mL) at room temperature and to the resulting solution was added N-bromosuccinimide (1.2 mmol). The resulting suspension was stirred at room temperature for 14 hours, then quenched with aqueous saturated sodium thiosulfate solution (20 mL). The reaction was concentrated in vacuo, and the resulting residue was diluted with ethyl acetate (75 mL). The aqueous layer was extracted with ethyl acetate (2*100 mL) and the combined organic layers were washed with aqueous 1N sodium bicarbonate solution (50 mL) and brine (50 mL), then dried over magnesium sulfate, filtered and concentrated in vacuo to provide title compound (87%), which was used further with or without purification.

### Step C: ethyl 6-benzyl-1-methyl-1,6-dihydroimidazo[4,5-d]pyrrolo[2,3-b]pyridine-8-carboxylate

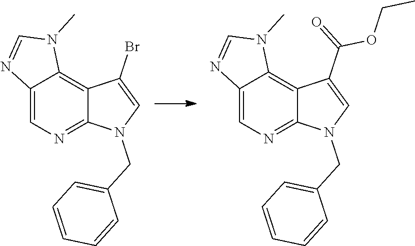 [SMILES: Cn1cnc2cnc3c(c(Br)cn3Cc3ccccc3)c21 ; CCOC(=O)c1cn(Cc2ccccc2)c2ncc3ncn(C)c3c12]

6-benzyl-8-bromo-1-methyl-1,6-dihydroimidazo[4,5-d]pyrrolo[2,3-b]pyridine (173 mmol) was added in dry tetrahydrofuran (500 mL) at −78° C. and n-butyl lithium (2.5 M solution in hexane, 487 mmol) was added over a period of 2 hours. The reaction mixture was stirred for another 30 minutes at −78° C. Ethyl chloroformate (186 mmol) was added over 30 minutes and the reaction mixture was stirred for 2 hours at −60° C. The temperature was slowly increased to 30° C. and mixture was allowed to stir for 12 hours at 30° C. The progress of the reaction was monitored by TLC. The reaction mixture was then quenched with saturated solution of ammonium chloride (150 mL) at 0° C. and the reaction mixture was extracted with ethyl acetate (3×300 mL). The combined organic layers were washed with water, dried over anhydrous sodium sulfate (50 g), filtered and concentrated under reduced pressure to afford a crude reaction mixture. The residue was purified by chromatography to provide the title compound (50%).

### Step D: 6-benzyl-N,1-dimethyl-1,6-dihydroimidazo [4,5-d]pyrrolo[2,3-b]pyridine-8-carboxamide

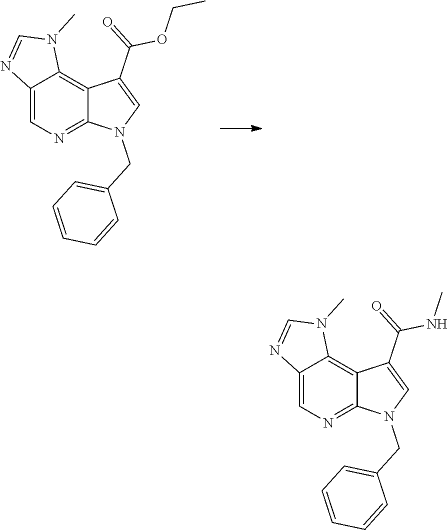 [SMILES: CCOC(=O)c1cn(Cc2ccccc2)c2ncc3ncn(C)c3c12 ; CNC(=O)c1cn(Cc2ccccc2)c2ncc3ncn(C)c3c12]

A solution of trimethylaluminium (2 M in toluene, 1.2 mmol) was added dropwise (exothermic) to a solution of methylamine (2 M in toluene, 1.2 mmol) in dioxane (7.5 mL) and the resulting mixture was stirred at room temperature for 1 h. Then a solution of ethyl 6-benzyl-1-methyl-1,6-dihydroimidazo[4,5-d]pyrrolo[2,3 -b]pyridine-8-carb oxylate (0.3 mmol) in dioxane (4 mL) was added. The resulting mixture was then heated at 85-95° C. for 3 h and then cooled to room temperature and then poured into water and extracted with MDC which was then washed with brine, dried over sodium sulfate and evaporated. Purification by chromatography (SiO2, MDC:MeOH=90:10) afforded the title compound as a white solid. (70%).

### Example 8: N-(propan-2-yl)-3,6-dihydroimidazo[4,5-d]pyrrolo[2,3- b]pyridine-8-carboxamide hydrochloride

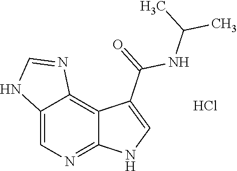 [SMILES: CC(C)NC(=O)c1c[nH]c2ncc3[nH]cnc3c12 ; Cl]

### Step A: 1-(1-benzyl-4-chloro-1H-pyrrolo[2,3-b]pyridin-3-yl)-2,2,2-trifluoroethanone

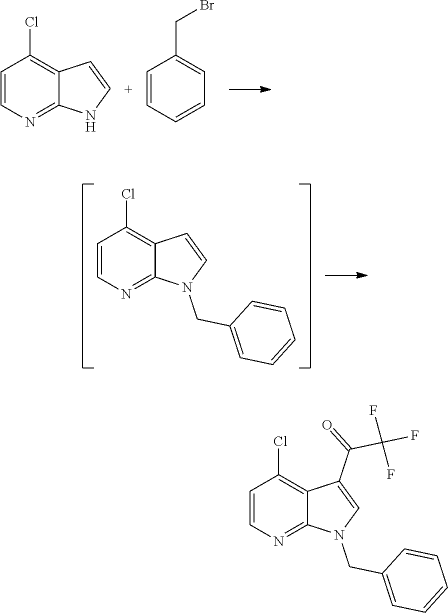

To a stirred suspension of sodium hydride (39.3 g, 1638.5 mmol, 60%) in dimethylacetamide (500mL) was added a solution of 4-chloro-7-aza indole (100g, 655.4 mmol) in dimethylacetamide (150 mL) at 0-5° C. followed by benzyl bromide (134.5 gm, 786.5 mmol). The resultant reaction mixture was stirred for 4.0 hours and then quenched with 100 ml of methanol followed by saturated ammonium chloride (500 mL) and extracted with ethylacetate. The organic layer was evaporated under reduced pressure to afford brown to yellow color liquid, 1-benzyl-4-chloro-1H-pyrrolo [2,3-b]pyridine (180 gm). The above compound was dissolved in in dimethylformamide (700 mL) and then was added trifluoroacetic anhydride (129.8 g, 618.0 mmol). The resulting reaction mixture was heated at 70-75° C. for 3.0 hours. Reaction mixture was cooled to 10-15° C. and was added ice cold water (500 mL) followed by saturated aqueous sodium bicarbonate. Reaction mixture was filtered and purified by using Isopropanol to obtain beige to light yellow color solid 1-(1-benzyl-4-chloro-1H-pyrrolo[2,3-b]pyridin-3-yl)-2,2,2-trifluoroethanone, (125.0 g89.6%).

¹HNMR (400 MHz, DMSO-d6): δ 9.03 (S,1H), δ 8.38 (m 1H), δ 7.46 (m, 1H), δ 7.34 (m,5H), δ 5.66 (S,2H)

### Step B: 1-(4-amino-1-benzyl-5-nitro-1H-pyrrolo[2,3-b]pyridin-3-yl)-2,2,2-trifluoroethanone

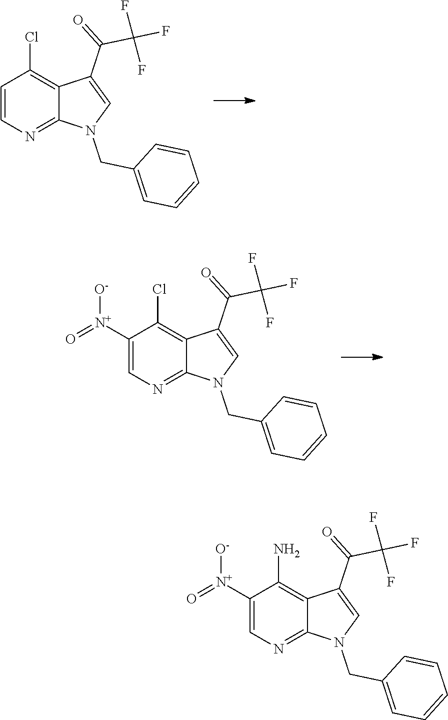 [SMILES: O=C(c1cn(Cc2ccccc2)c2nccc(Cl)c12)C(F)(F)F ; O=C(c1cn(Cc2ccccc2)c2ncc([N+](=O)[O-])c(Cl)c12)C(F)(F)F ; Nc1c([N+](=O)[O-])cnc2c1c(C(=O)C(F)(F)F)cn2Cc1ccccc1]

To a stirred solution of 1-(1-benzyl-4-chloro-1H-pyrrolo[2,3-b]pyridin-3-yl)-2,2,2-trifluoroethanone (100 g, 295.2 mmol) in dichloromethane (2500mL) was added tetrabutylammonium nitrate (224.7 g, 738.0 mmol) in portions followed by dropwise addition of trifluoroacetic anhydride (155 g, 738.0 mmol) at 0° C. The reaction mixture was stirred for 5.0 hours at room temperature. Organic layer was washed with water and concentrated under reduced pressure to afford yellow solid, 1-(1-benzyl-4-chloro-5-nitro-1H-pyrrolo[2,3-b]pyridin-3-yl)-2,2,2-trifluoroethanone (100 gm 88.5%).

To a stirred solution of 1-(1-benzyl-4-chloro-5-nitro-1H-pyrrolo [2,3-b]pyridin-3-yl)-2,2,2-trifluoroethanone (100 g, 260.6mmo1) in dichloromethane (500 mL) was purged ammonia gas till completion of reaction on TLC. Solvent was removed under reduced pressure. The residue was dissolved in dichloromethane (300 ml) , cooled to 5-10° C. and filtered. The obtained wet solid was dried under vacuum to afford yellow solid, 1-(4-amino-1-benzyl-5-nitro-1H-pyrrolo[2,3-b]pyridin-3-yl)-2,2,2-trifluoroethanone (85 gm ,89. 5%)

¹HNMR (400 MHz, DMSO-d6): δ 9.04 (S,1H), δ 8.94(m 1H), δ 8.73 (S, 1H), δ 7.30 (m,6H), δ 5.57 (S,2H)

### Step C: 1-(4,5-diamino-1-benzyl-1H-pyrrolo[2,3-b]pyridin-3-yl)-2,2,2-trifluoroethanone

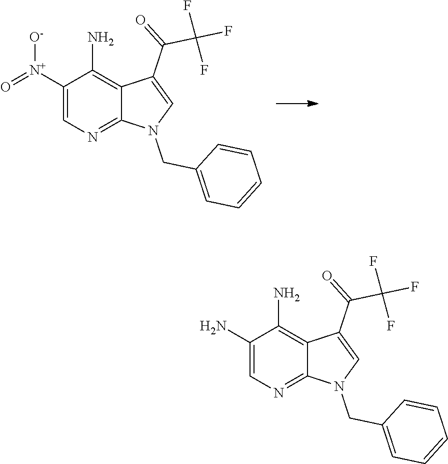 [SMILES: Nc1c([N+](=O)[O-])cnc2c1c(C(=O)C(F)(F)F)cn2Cc1ccccc1 ; Nc1cnc2c(c(C(=O)C(F)(F)F)cn2Cc2ccccc2)c1N]

To a stirred solution of 1-(4-amino-1-b enzyl-5-nitro-1H-pyrrolo[2,3-b]pyridin-3-yl)-2,2,2-trifluoroethanone (85 g, 233.3 mmol) in mixture of methanol: tetrahydrofuran (1500 mL, 1:0.5) was added Raney Nickel (21.2 g 25.0% w/w) followed by dropwise addition of hydrazine hydrate (59.5 ml, 0.70 w/v) and reaction mixture was stirred for 1.0 hours at room temperature. After completion, reaction mixture was filtered through hyflo bed and washed with methanol (400 mL). The filtrate was concentrated under reduced pressure and obtained was purified by water (500 mL), filtered and dried under reduced pressure to afford brown color solid 1-(4,5-diamino-1-benzyl-1H-pyrrolo[2,3-b]pyridin-3-yl)-2,2,2-trifluoroethanone (71.0gm 90.8%)

¹HNMR (400 MHz, DMSO-d6): δ 8.59 (d,1H), δ 7.66 (s 1H), δ 7.33 (m, 4H), δ 7.26 (m,1H), δ 6.56 (s,2H), δ 5.45 (s,2H), δ 4.47 (s,2H)

### Step D: 1-(6-benzyl-3,6-dihydroimidazo[4,5-d]pyrrolo[2,3-b]pyridin-8-yl)-2,2,2-trifluoroethanone

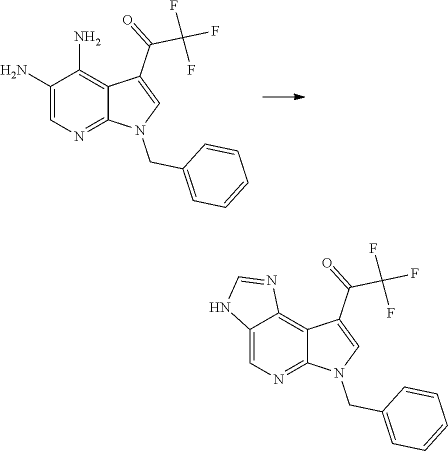 [SMILES: Nc1cnc2c(c(C(=O)C(F)(F)F)cn2Cc2ccccc2)c1N ; O=C(c1cn(Cc2ccccc2)c2ncc3[nH]cnc3c12)C(F)(F)F]

To stirred suspension of 1-(4,5-diamino-1-benzyl-1H-pyrrolo[2,3-b]pyridin-3-yl)-2,2,2-trifluoroethanone (70 g, 209.4 mmol) in toluene (700 mL) was added triethylorthoformate (96.7 mL, 418.8 mmol) and para-toulenesulfonic acid monohydrate (8.0 g 41.88 mmol). The resulting reaction mixture was heated at 80-85° C. for 5.0 hours. After completion, reaction mixture was concentrated under reduced pressure. To the obtained residue was added water (700 mL), stirred at room temperature and filtered an dried to afford 1-(6-benzyl-3,6-dihydroimidazo[4,5-d]pyrrolo[2,3-b]pyridin-8-yl)-2,2,2-trifluoroethanone (65 gm,90.1%).

¹HNMR (400 MHz, DMSO-d6): δ 12.51(bs,1H), δ 8.89 (m 2H), δ 8.29 (t, 1H), δ 7.31 (m,5H), δ 5.72(s,2H)

### Step E: 6-benzyl-3,6-dihydroimidazo[4,5-d]pyrrolo[2,3-b]pyridine-8-carboxylic acid

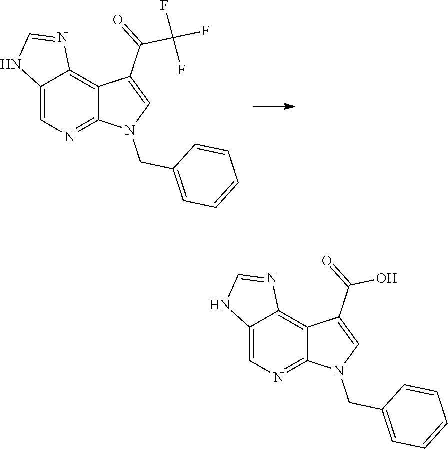 [SMILES: O=C(c1cn(Cc2ccccc2)c2ncc3[nH]cnc3c12)C(F)(F)F ; O=C(O)c1cn(Cc2ccccc2)c2ncc3[nH]cnc3c12]

To the solution of sodium hydroxide (151 gm,3775 mmol) in water (945 mL) was added 1-(6-benzyl-3,6-dihydroimidazo[4,5-d]pyrrolo[2,3-b]pyridin-8-yl)-2,2,2-trifluoroethanone (65 gm,188.8 mmol), reaction mass was heated at 80-85° C. for 5.0 hours. After completion, reaction mixture was diluted with water followed by dilute HCl and filtered. The obtained wet cake was dried under vacuum to afford beige to light brown color solid 6-benzyl-3,6-dihydroimidazo[4,5-d]pyrrolo[2,3-b]pyridine-8-carboxylic acid, (50 gm,90.5%)

¹HNMR (400 MHz, DMSO-d6): δ 12.2(s,1H), δ 8.75(s, 1H), δ 8.22(s,1H), δ 8.17 (s, 1H) δ 7.27(m,5H), δ 5.61(s,2H)

### Step F: 3,6-dihydroimidazo[4,5-d]pyrrolo[2,3-b]pyridine-8-carboxylic acid

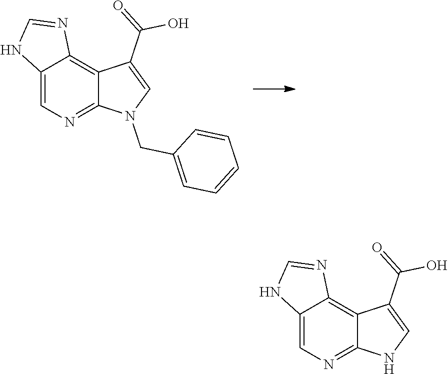 [SMILES: O=C(O)c1cn(Cc2ccccc2)c2ncc3[nH]cnc3c12 ; O=C(O)c1c[nH]c2ncc3[nH]cnc3c12]

To the solution of liquid ammonia (750 mL) was added sodium metal (32.8 g, 1368.5 mmol) lot wise. To the resulting reaction mixture, was added tertiary butanol (50 mL), Tetrahydrofuran (500 mL) followed by 6-benzyl-3,6-dihydroimidazo[4,5-d]pyrrolo[2,3-b]pyridine-8-carboxylic acid (50 g, 171.1 mmol). Then reaction mixture was stirred at −60° C. to −30° C. for 4.0 hr and quenched with methanol (50 mL) and water (50 mL). Solvent was evaporated under reduced pressure. Then was added water (100 ml) to the residue followed by HC1 and stirred. The reaction mixture was filtered and wet solid was dried under reduced pressure to afford beige color solid 3,6-dihydroimidazo[4,5-d]pyrrolo[2,3-b]pyridine-8-carboxylic acid, (32 gm,91.4%).

¹HNMR (400 MHz, DMSO-d6): δ 12.39 (bs,1H), δ 12.07 (bs 1H), δ 8.66(s, 1H), δ 8.14(d,1H), δ 7.94 (s,1H)

### Step G: N-(propan-2-yl)-3,6-dihydroimidazo [4,5-d]pyrrolo [2,3-b]pyridine-8-carboxamide

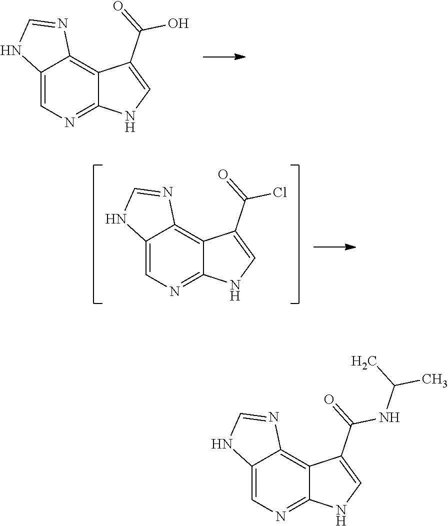

To a solution of 3,6-dihydroimidazo[4,5-d]pyrrolo[2,3-b]pyridine-8-carboxylic acid (30.0 g, 148.4 mmol) in dimethylformamide (15 mL) was added thionyl chloride (300 mL) and reaction mixture was heated to 65-70° C. and stirred for 4.0 hours. After completion, reaction mixture was concentrated under reduced pressure to obtain acid chloride (30 g) which was used as such for further reaction.Above acid chloride derivative(30 g) was taken into dichloromethane (300 mL), cooled to 5-10° C. and added isopropyl amine (300 mL). The resulting reaction mixture was stirred for 5.0 hours at room temperature. After completion, reaction mass was concentrated under reduced pressure, added water (150 mL) and filtered.

The obtained wet solid was dried under vacuum to afford beige to off white color solid N-(propan-2-yl)-3,6-dihydroimidazo[4,5-d]pyrrolo[2,3-b]pyridine-8-carboxamide (29.0 gm,80.3%)

¹HNMR (400 MHz, DMSO-d6): δ 13.04 (bs,1H), δ 12.19 (t, 1H), δ 10.03(d,1H), δ 8.59(d,2H), δ 8.07(m,1H), δ 4.19(m,1H), δ 1.22(td,6H)

### Step H: N-(propan-2-yl)-3,6-dihydroimidazo[4,5-d]pyrrolo[2,3-b]pyridine-8-carboxamide hydrochloride

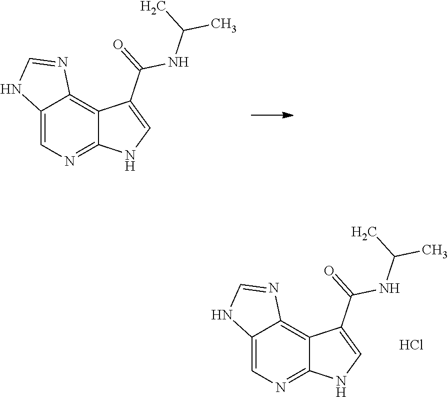 [SMILES: CC(C)NC(=O)c1c[nH]c2ncc3[nH]cnc3c12 ; CC(C)NC(=O)c1c[nH]c2ncc3[nH]cnc3c12 ; Cl]

To a solution of N-(propan-2-yl)-3,6-dihydroimidazo[4,5-d]pyrrolo[2,3-b]pyridine-8-carboxamide (25.0 g, 102.8 mmol) in isopropanol(175 mL) was added solution of HCl in isopropanol at 10-15° C. The resultant reaction mixture was stirred at 50-55° C. 2.0 hr. After completion, reaction mixture was concentrated under reduced pressure. To the residue obtained was added water (250 mL) and stirred for 1.0 hour at room temperature, filtered and dried under vacuumto afford beige color solid N-(propan-2-yl)-3,6-dihydroimidazo[4,5-d]pyrrolo[2,3-b]pyridine-8-carboxamide hydrochloride, (25 gm, 87.0%)

¹HNMR (400 MHz, DMSO-d6): δ 13.66 (bs,1H), δ 12.87(bs, 1H), δ 9.26(s,1H), δ 8.86(s,1H), δ 8.57(d,2H), δ 4.81 (bs 1H), δ 4.22(qd,1H), δ 1.25(d,6H)

### Example 9: N-ethyl-3,6-dihydroimidazo[4,5-cl]pyrrolo[2,3-b]pyridine-8-carboxamide

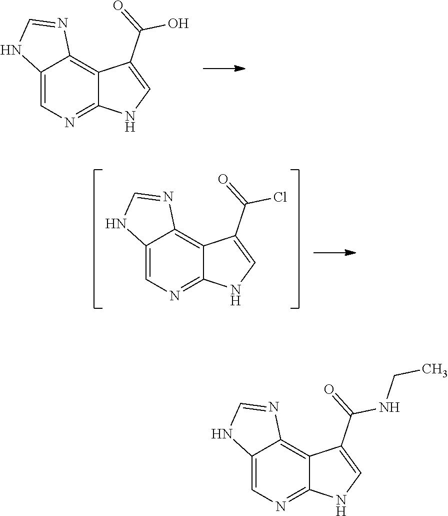

To a solution of 3,6-dihydroimidazo[4,5-d]pyrrolo[2,3-b]pyridine-8-carboxylic acid (30.0 g, 148.4 mmol) in dimethylformamide (15 mL) was added thionyl chloride (300 mL) and reaction mixture was heated to 65-70° C. and stirred for 4.0 hours. After completion, reaction mixture was concentrated under reduced pressure to obtain acid chloride (32 g) which was used as such for further reaction.

Above acid chloride derivative (32 g) was dissolved indichloromethane (300 mL), cooled to 5-10° C. and added ethylamine amine (300 mL). The resulting reaction mixture was stirred for 5.0 hours at room temperature. After completion, reaction mass was concentrated under reduced pressure, added water (150 mL) and filtered.

The obtained wet solid was dried under vacuum to afford beige color solid N-ethyl-3,6-dihydroimidazo[4,5-d]pyrrolo[2,3-b]pyridine-8-carboxamide (27.0 gm,79.4%)

¹HNMR (400 MHz, DMSO-d6): δ 13.24 (bs,1H), δ 12.4 (t, 1H), δ 10.2(d,1H), δ 8.62(d,2H), δ 8.23(m,2H), δ 3.2 (q,2H), δ 1.22(t,3H)

### Example 10: N-propyl-3,6-dihydroimidazo[4,5-d]pyrrolo[2,3-b]pyridine-8-carboxamide

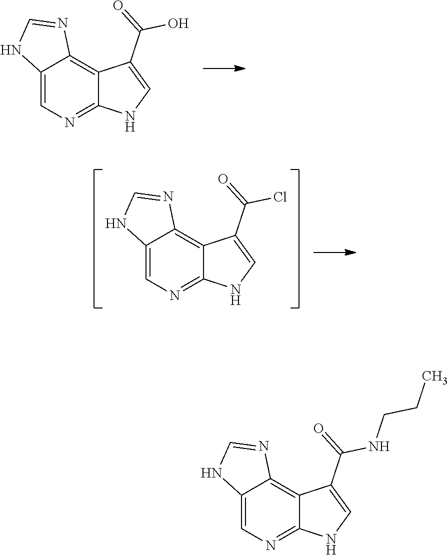

To a solution of 3,6-dihydroimidazo[4,5-d]pyrrolo[2,3-b]pyridine-8-carboxylic acid ( 30.0 g, 148.4 mmol) in dimethylformamide (15 mL) was added thionyl chloride (300 mL) and reaction mixture was heated to 65-70° C. and stirred for 4.0 hours. After completion, reaction mixture was concentrated under reduced pressure to obtain acid chloride(31 g) which was used as such for further reaction.

Above acid chloride derivative (31 g) was dissolved indichloromethane (300 mL), cooled to 5-10° C. and added n-propyl amine (300 mL). The resulting reaction mixture was stirred for 5.0 hours at room temperature. After completion, reaction mass was concentrated under reduced pressure, added water (150 mL) and filtered.

The obtained wet solid was dried under vacuum to afford beige color solid N-propyl-3,6-dihydroimidazo[4,5-d]pyrrolo[2,3-b]pyridine-8-carboxamide, (30.0 gm, 83.3%)

¹HNMR (400 MHz, DMSO-d6): δ 12.15 (t, 1H), δ 10.24(d,1H), δ 8.24(d,2H), δ 8.15(m,2H), δ 3.22 (t,2H), δ 1.6(m,2H) δ 1.2(t,3H)

### Example 11: N-butyl-3,6-dihydroimidazo[4,5-d]pyrrolo[2,3-b]pyridine-8-carboxamide

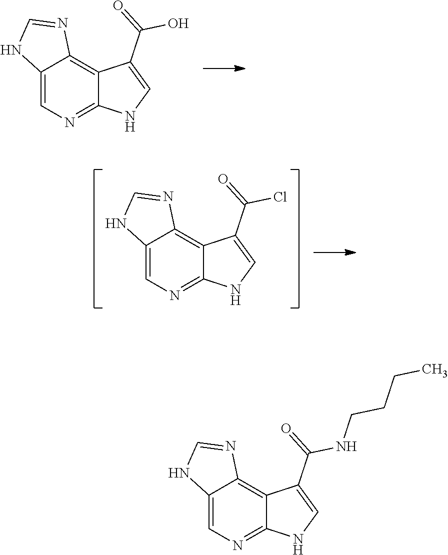

To a solution of 3,6-dihydroimidazo[4,5-d]pyrrolo[2,3-b]pyridine-8-carboxylic acid ( 30.0 g, 148.4 mmol) in dimethylformamide (15 mL) was added thionyl chloride (300 mL) and reaction mixture was heated to 65-70° C. and stirred for 4.0 hours. After completion, reaction mixture was concentrated under reduced pressure to obtain acid chloride(33 g) which was used as such for further reaction.

Above acid chloride derivative (33 g) was dissolved indichloromethane (300 mL), cooled to 5-10° C. and added and added n-butylamine(300 mL). The resulting reaction mixture was stirred for 5.0 hours at room temperature. After completion, reaction mass was concentrated under reduced pressure, added water (150 mL) and filtered.

The obtained wet solid was dried under vacuum to afford beige color solid N-butyl-3,6-dihydroimidazo[4,5-d]pyrrolo[2,3-b]pyridine-8-carboxamide 32.0 g (83.8%)

¹HNMR (400 MHz, DMSO-d6): δ 12.12(t, 1H), δ 10.4(d,1H), δ 8.25(d,2H), δ 8.3(m,2H), 6 3.2 (t,2H). δ 1.6 (m, 4H), δ 1.2 (t, 2H)

### Example 12: N-methyl-3,6-dihydroimidazo[4,5-d]pyrrolo[2,3-b]pyridine-8-carboxamide

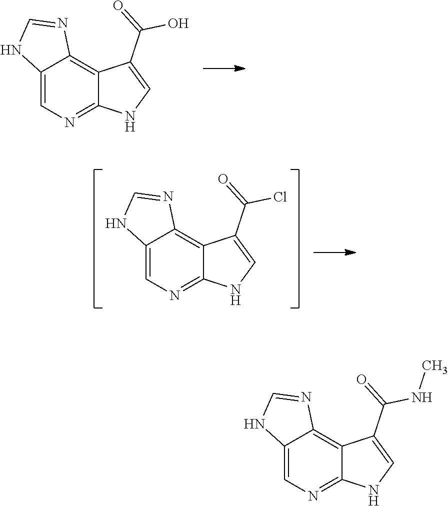

To a solution of 3,6-dihydroimidazo[4,5-d]pyrrolo[2,3-b]pyridine-8-carboxylic acid ( 30.0 g, 148.4 mmol) in dimethylformamide (15 mL) was added thionyl chloride (300 mL) and reaction mixture was heated to 65-70° C. and stirred for 4.0 hours. After completion, reaction mixture was concentrated under reduced pressure to obtain acid chloride (28 g) which was used as such for further reaction.

Above acid chloride (28 g) was dissolved in dichloromethane (300 mL), cooled to 5-10° C. and added methylamine hydrochloride (51.0 g 757.0mmo1). The resulting reaction mixture was stirred for 5.0 hours at room temperature. After completion, reaction mass was concentrated under reduced pressure, added water (150 mL) and filtered.

The obtained wet solid was dried under vacuum to afford beige color solid N-methyl-3,6-dihydroimidazo[4,5-d]pyrrolo[2,3-b]pyridine-8-carboxamide, (25.0 g 78.4%)

¹HNMR (400 MHz, DMSO-d6): δ 12.2(t, 1H), δ 10.2(d,1H), δ 8.3(d,2H), δ 8.4(m,2H), δ 3.3 (S,3H)

### Example 13: 3,6-dihydroimidazo[4,5-d]pyrrolo[2,3-b]pyridine-8-carboxamide

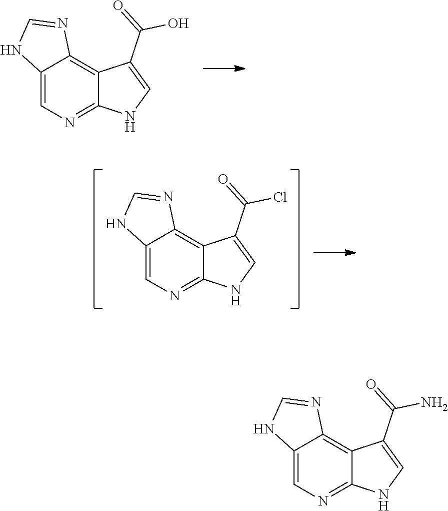

To a solution of 3,6-dihydroimidazo[4,5-d]pyrrolo[2,3-b]pyridine-8-carboxylic acid (30.0 g, 148.4 mmol) in dimethylformamide (15 mL) was added thionyl chloride (300 mL) and reaction mixture was heated to 65-70° C. and stirred for 4.0 hours. After completion, reaction mixture was concentrated under reduced pressure to obtain acid chloride (25 g) which was used as such for further reaction.

Above acid chloride (28 g) was dissolved in dichloromethane (300 mL), cooled to 5-10° C. and purgeammonia gas till completion of reaction on TLC. The resulting reaction mixture was stirred for 5.0 hours at room temperature. After completion, reaction mass was concentrated under reduced pressure, added water (150 mL) and filtered.

The obtained wet solid was dried under vacuum to afford beige color solid 3,6-dihydroimidazo[4,5-d]pyrrolo[2,3-b]pyridine-8-carboxamide, (22.0 gm,73.7%)

¹HNMR (400 MHz, DMSO-d6): δ 13.3(bs,1H), δ 12.2(bs, 1H), δ 10.2(d,1H), δ 8.3(d,1H), δ 8.4(m,1H), δ 7.3 (bs,2H).

1. A compound of formula (I) 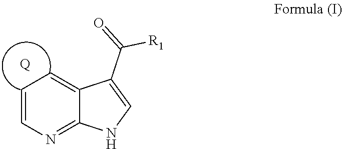
or a pharmaceutically acceptable salt thereof;
wherein Q is a group of formula Q1 or Q2; 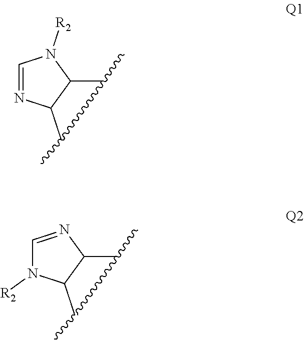 [symbol]
(wavy bond) represents the points of attachment;
R₁ is —NR^{a}R^{b},
R₂ is hydrogen or a C₁-C₁₀ alkyl group;
R^{a} and R^{b} independently represent hydrogen or a C₁-C₁₀ alkyl group.

2. The compound of claim 1, wherein Q is Q1 and R₂ represents hydrogen or a C₁-C₁₀ alkyl group.

3. The compound of claim 1, wherein Q is Q2 and R₂ represents hydrogen or a C₁-C₁₀ alkyl group.

4. The compound of claim 1, wherein R₁ is —NHR^{a} or —NHR^{b}.

5. (canceled)

6. The compound of claim 1, wherein R₂ is methyl or hydrogen.

7. (canceled)

8. The compound of claim 1, wherein R^{a} is methyl, ethyl, propyl, or isopropyl; R^{b} is hydrogen and R₂ is methyl.

9. (canceled)

10. (canceled)

11. (canceled)

12. The compound of claim 1, wherein R^{a} is methyl, ethyl, propyl, or isopropyl; and R^{b} and R2 are hydrogen.

13. (canceled)

14. (canceled)

15. (canceled)

16. The compound of claim 1, wherein the compound is selected from the group consisting of: N, 1-dimethyl-1,6-dihydroimidazo[4,5-d]pyrrolo[2,3-b ]pyridine-8-carboxamide; N-ethyl-1-methyl-1,6-dihydroimidazo[4,5-d]pyrrolo[2,3-b ]pyridine-8-carboxamide; 1-methyl-N-propyl-1,6-dihydroimidazo[4,5-d]pyrrolo[2,3-b ]pyridine-8-carboxamide; 1-methyl-N-(propan-2-yl)-1,6-dihydroimidazo[4,5-d]pyrrolo[2,3-b]pyridine-8-carboxamide; N-methyl-1,6-dihydroimidazo[4,5-d]pyrrolo[2,3-b]pyridine-8-carboxamide; N-ethyl-1,6-dihydroimidazo[4,5-d]pyrrolo[2,3-b]pyridine-8-carboxamide; N-(propan-2-yl)-1,6-dihydroimidazo[4, 5-d]pyrrolo[2,3-b]pyridine-8-carboxamide; N,3-dimethyl-3,6-dihydroimidazo[4 ,5-d]pyrrolo[2,3-b]pyridine-8-carboxamide; N-ethyl-3-methyl-3, 6-dihydroim idazo[4, 5-d]pyrrolo[2,3-b]pyridine-8-carboxamide; 3-methyl-N-propyl-3,6-dihydroimidazo[4,5-d]pyrrolo[2,3-b]pyridine-8-carboxamide; 3-methyl-N-(propan-2-yl)-3,6-dihydroimidazo[4,5-d]pyrrolo[2,3-b]pyridine-8-carboxamide; N-methyl-3,6-dihydroimidazo[4, 5-d]pyrrolo[2,3-b]pyridine-8-carboxamide; N-ethyl-3,6-dihydroimidazo[4,5-d]pyrrolo[2,3-b]pyridine-8-carboxamide; and N-(propan-2-yl)-3,6-dihydroimidazo[4,5-d]pyrrolo[2,3-b]pyridine-8-carboxamide; or a pharmaceutically acceptable salt thereof.

17. The compound of claim 1, wherein the compound is selected from the group consisting of: N, 1-dimethyl-1,6-dihydroimidazo[4,5-d]pyrrolo[2,3-b]pyridine-8-carboxamide; N-ethyl-1-methyl-1,6-dihydroimidazo[4,5-d]pyrrolo[2,3-b]pyridine-8-carboxamide; 1-methyl-N-propyl-1,6-dihydroimidazo[4,5-d]pyrrolo[2,3-b]pyridine-8-carboxamide; 1-methyl-N-(propan-2-yl)-1,6-dihydroimidazo[4,5-d]pyrrolo[2,3-b]pyridine-8-carboxamide; N-methyl-1,6-dihydroimidazo[4,5-d]pyrrolo[2,3-b]pyridine-8-carboxamide; N-ethyl-1,6-dihydroimidazo[4,5-d]pyrrolo[2,3-b]pyridine-8-carboxamide; N-(propan-2-yl)-1,6-dihydroimidazo[4, 5-d]pyrrolo[2,3-b]pyridine-8-carboxamide; N,3-dimethyl-3,6-dihydroimidazo[4 ,5-d]pyrrolo[2,3-b]pyridine-8-carboxamide; N-ethyl-3-methyl-3,6-dihydroim idazo[4, 5-d]pyrrolo[2,3-b ]pyridine-8-carboxamide; 3-methyl-N-propyl-3,6-dihydroimidazo[4,5-d]pyrrolo[2,3-b]pyridine-8-carboxamide; 3-methyl-N-(propan-2-yl)-3,6-dihydroimidazo[4,5-d]pyrrolo[2,3-b]pyridine-8-carboxamide; N-methyl-3,6-dihydroimidazo[4, 5-d]pyrrolo[2,3-b]pyridine-8-carboxamide; N-ethyl-3,6-dihydroimidazo[4,5-d]pyrrolo[2,3-b]pyridine-8-carboxamide; and N-(propan-2-yl)-3,6-dihydroimidazo[4,5-d]pyrrolo[2,3-b]pyridine-8-carboxamide.

18. The compound according to claim 16, wherein the compound is N-(propan-2-yl)-1,6-dihydroimidazo[4,5-d]pyrrolo[2,3-]pyridine-8-carboxamide hydrochloride.

19. The compound according to claim 17, wherein the compound is N-(propan-2-yl)-3,6-dihydroimidazo[4,5-d]pyrrolo[2,3-b]pyridine-8-carboxamide hydrochloride.

20. A pharmaceutical composition, the pharmaceutical composition comprising a compound according to claim 1.

21. The pharmaceutical composition of claim 20, wherein the pharmaceutical composition further comprises a pharmaceutically acceptable excipient.

22. The pharmaceutical composition of claim 20 for use in therapy or in the treatment or prevention of a disease or condition that is caused by an abnormal functioning of a kinase.

23. (canceled)

24. The pharmaceutical composition of claim 22, wherein the kinase is a Janus kinase.

25. The pharmaceutical composition of claim 24, wherein the disease or condition that is caused by an abnormal functioning of a Janus kinase is selected from the group consisting of a proliferative disease, a disease involving impairment of cartilage turnover, a disease involving the anabolic stimulation of chondrocytes, an autoimmune disease, congenital cartilage malformation(s), an inflammatory condition and transplantation rejection.

26. A method manufacturing a medicament for use in the treatment or prevention of a disease or condition that is caused by an abnormal functioning of a kinase, the method comprising incorporating a compound of claim 1 into the medicament.

27. The method of claim 26, wherein the kinase is a Janus kinase; wherein the disease or condition that is caused by an abnormal functioning of Janus kinase is selected from the group consisting of a proliferative disease, a disease involving impairment of cartilage turnover, a disease involving the anabolic stimulation of chondrocytes, an autoimmune disease, congenital cartilage malformation(s), an inflammatory condition and transplantation rejection.

28. (canceled)

29. A method of treating or preventing a disease or condition that is caused by an abnormal functioning of a kinase in a subject need thereof, the method comprising administering to the subject a therapeutically effective amount of a compound according to claim 1 to 19.

30. The method of claim 29, wherein the kinase is a Janus kinase; wherein the disease or condition that is caused by an abnormal functioning of a Janus kinase is selected from the group consisting of a proliferative disease, a disease involving impairment of cartilage turnover, a disease involving the anabolic stimulation of chondrocytes, an autoimmune disease, congenital cartilage malformation(s), an inflammatory condition and transplantation rejection.

31. (canceled)
